(19) United States
(12) Patent Application Publication
(10) Pub. No.: US 2025/0113999 A1
(43) Pub. Date: Apr. 10, 2025

(54) SYSTEMS, APPARATUS, ARTICLES OF MANUFACTURE, AND METHODS FOR GAZE ANGLE TRIGGERED FUNDUS IMAGING

(71) Applicant: Tesseract Health, Inc., Guilford, CT (US)

(72) Inventors: Luka Djapic, Branford, CT (US); Brett J. Gyarfas, Aptos, CA (US); Jose Bscheider, Southbury, CT (US); David Morris Lion, Seattle, WA (US); Andrew Homyk, Belmont, CA (US); Georg Schuele, Portola Valley, CA (US); Alex Krasner, Princeton, NJ (US); Krishna Adithya Venkatesh, Redwood City, CA (US); Noah Wilson, Santa Cruz, CA (US); Tushar Kulkarni, West Hartford, CT (US)

(73) Assignee: Tesseract Health, Inc., Guilford, CT (US)

(21) Appl. No.: 18/906,756

(22) Filed: Oct. 4, 2024

Related U.S. Application Data
(60) Provisional application No. 63/702,518, filed on Oct. 2, 2024.
(60) Provisional application No. 63/588,609, filed on Oct. 6, 2023.

(51) Int. Cl. A61B 3/12; A61B 3/00; A61B 3/14; G06T 7/00; G06T 7/13; G06T 7/136; G06T 7/62; G06T 7/80

(52) U.S. Cl. CPC A61B 3/12; A61B 3/0025; A61B 3/113; A61B 3/14; G06T 7/0012; G06T 7/13; G06T 7/136; G06T 7/62; G06T 7/80; G06T 2207/10012; G06T 2207/20081; G06T 2207/20172; G06T 2207/30041

(57) ABSTRACT

The techniques described herein relate to systems, apparatus, articles of manufacture, and methods for gaze angle triggered fundus imaging. An example method includes detecting a pupil of a subject in a stereo image, controlling at least one actuator to align an imaging path of a fundus camera with the pupil after detecting the pupil in the stereo image, and capturing a fundus image of a retina of the subject at a gaze angle after determining that the pupil is oriented towards a target direction based on the gaze angle associated with the pupil in the image.

### CROSS-REFERENCE TO RELATED APPLICATIONS

This application claims priority under 35 U.S.C. § 119(e) to: U.S. Provisional Application Ser. No. 63/702,518, filed Oct. 2, 2024, under Attorney Docket No. T0753.70042US00, and entitled “SYSTEMS, APPARATUS, ARTICLES OF MANUFACTURE, AND METHODS FOR GAZE ANGLE TRIGGERED FUNDUS IMAGING,” and to U.S. Provisional Application Ser. No. 63/588,609, filed Oct. 6, 2023, under Attorney Docket No. T0753.70036US00, and entitled “SYSTEMS, APPARATUS, ARTICLES OF MANUFACTURE, AND METHODS FOR GAZE ANGLE TRIGGERED FUNDUS IMAGING,” each application of which is incorporated by reference herein in its entirety.

### FIELD OF THE DISCLOSURE

The present disclosure relates generally to imaging an eye fundus and, more particularly, to systems, apparatus, articles of manufacture, and methods for gaze angle triggered fundus imaging.

### BACKGROUND

Fundus photography is capturing an image of an inner lining of a retina of an eye, such as an eye of a human subject, with a fundus camera. The inner lining of the retina may also be referred to as the fundus of the eye. The fundus camera may capture the image by causing the illumination and reflectance of the retina to occur through a common optical path, such as a pupil of the eye. A medical professional such as an ophthalmologist may use the image to diagnose and/or develop a treatment plan for various ocular diseases.

### SUMMARY OF THE DISCLOSURE

In accordance with the disclosed subject matter, systems, apparatus, articles of manufacture, and methods are provided for gaze angle triggered fundus imaging.

Some embodiments relate to an example method for triggering fundus imaging. The example method comprises detecting a pupil of a subject in an image, controlling at least one actuator to align an imaging path of a fundus camera with the pupil after detecting the pupil in the stereo image, and capturing a fundus image of a retina of the subject at a gaze angle after determining that the pupil is oriented towards an intended fixation target direction based on the gaze angle associated with the pupil in the image.

Some embodiments relate to an example apparatus comprising at least one memory storing machine-readable instructions, and at least one processor configured to execute the machine-readable instructions to perform at least the aforementioned method.

Some aspects relate to at least one example non-transitory computer-readable storage medium comprising instructions that, when executed, cause at least one processor to perform at least the aforementioned method.

Some aspects relate to an example fundus camera system comprising a three-dimensional visualization system, a fundus camera, at least one memory storing machine-readable instructions, and at least one processor configured to execute the machine-readable instructions to perform at least the aforementioned method.

The foregoing summary is not intended to be limiting. Moreover, various aspects of the present disclosure may be implemented alone or in combination with other aspects.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is an illustration of an example fundus camera system including an example fundus camera in communication with an example fundus computing system and configured to capture an image of a retina of a subject, according to some embodiments.

FIG. 2 is a block diagram of example implementations of the fundus camera system and the fundus computing system of FIG. 1, according to some embodiments.

FIG. 3A is an image of a human subject in which a pupil is not detected, according to some embodiments.

FIG. 3B is another image of the human subject of FIG. 3A in which a pupil is detected in a first frame of the image, according to some embodiments.

FIG. 3C is yet another image of the human subject of FIG. 3A in which a pupil is detected in a first frame and a second frame of the image, according to some embodiments.

FIG. 4A is an image of a human subject in which a pupil is detected, according to some embodiments.

FIG. 4B depicts a digital representation of the pupil of FIG. 4A, according to some embodiments.

FIG. 4C depicts a digital representation of a border of the pupil of FIG. 4A, according to some embodiments.

FIG. 5A is a first stereo image that may be captured by the example fundus camera system of FIG. 1 using a first set of coordinates, according to some embodiments.

FIG. 5B is a second stereo image that may be captured by the example fundus camera system of FIG. 1 using a second set of coordinates corrected from the first set of coordinates of FIG. 5A, according to some embodiments.

FIG. 6A is an illustration of a first stereo image of a subject's pupil in which the subject is looking in a first direction, according to some embodiments.

FIG. 6B is an illustration of a second stereo image of a subject's pupil in which the subject is looking in a second direction, according to some embodiments.

FIG. 6C is an illustration of a first stereo image of a subject's pupil in which the subject is looking in a third direction, according to some embodiments.

FIG. 7 is a flowchart representative of an example process and/or example machine-readable instructions that may be executed by processor circuitry to implement the fundus camera system of FIGS. 1 and/or 2 to capture a fundus image of a retina of a subject based on a gaze angle associated with a pupil, according to some embodiments.

FIG. 8 is a flowchart representative of an example process and/or example machine-readable instructions that may be executed by processor circuitry to implement the fundus camera system of FIGS. 1 and/or 2 to perform gaze angle triggered fundus imaging, according to some embodiments.

FIG. 9 is a flowchart representative of an example process and/or example machine-readable instructions that may be executed by processor circuitry to implement the fundus camera system of FIGS. 1 and/or 2 to perform pupil detection, according to some embodiments.

FIG. 10 is a flowchart representative of an example process and/or example machine-readable instructions that may be executed by processor circuitry to implement the fundus camera system of FIGS. 1 and/or 2 to perform pupil alignment, according to some embodiments.

FIG. 11 is a flowchart representative of an example process and/or example machine-readable instructions that may be executed by processor circuitry to implement the fundus camera system of FIGS. 1 and/or 2 to perform gaze angle verification, according to some embodiments.

FIG. 12 is a flowchart representative of an example process and/or example machine-readable instructions that may be executed by processor circuitry to implement the fundus camera system of FIGS. 1 and/or 2 to train and execute a machine-learning model to generate output(s) including at least one of a detection of a pupil or a gaze angle of the pupil with respect to a fundus camera, according to some embodiments.

FIG. 13 is a flowchart representative of an example process and/or example machine-readable instructions that may be executed by processor circuitry to implement the fundus camera system of FIGS. 1 and/or 2 to output feedback to a subject to improve pupil detection, according to some embodiments.

FIG. 14 is an example electronic platform structured to execute the machine-readable instructions of FIGS. 7, 8, 9, 10, 11, 12, and/or 13 to implement the fundus camera system of FIGS. 1 and/or 2, according to some embodiments.

### DETAILED DESCRIPTION

The present application generally provides techniques for gaze angle triggered fundus imaging. Fundus photography, and/or, more generally, fundus imaging, is capturing an image and/or photograph of an inner lining of a retina of an eye, such as an eye of a human subject, with a fundus camera. The inner lining of the retina may also be referred to as the fundus of the eye or simply the fundus. The fundus camera may record images of the condition of the fundus to document the presence (or absence) of disorders and monitor their change over time.

A fundus camera (also known as a retinal camera) may be implemented by a specialized, low power microscope with an attached camera designed and/or configured to image the interior surface of the eye, including the retina, retinal vasculature, optic disc, macula, and posterior pole (i.e., the fundus). To facilitate fundus imaging, a human subject's eyes may be dilated before image capture, such as by administering eye drops in the pupil of each eye to be imaged. Widening (i.e., dilating) a patient's pupil reduces the likelihood of clipping the illumination and detection beams while allowing for a larger spatial separation of these two paths, which also assists with mitigating and/or reducing image artifacts such as halo and glare. The increase of imaging angle of observation enables an operator controlling the fundus camera to image an increased area of the back of the eye and have a larger view thereof. Non-limiting examples of a fundus camera operator include a medical doctor (e.g., an ophthalmologist), an optometrist, an optician, and a technician.

Typically during fundus imaging, a human subject may be instructed to sit in front of the fundus camera with the subject's chin resting on a chin rest (e.g., a fundus camera attachment) and the subject's forehead against a bar or other support structure. The fundus camera operator may focus and align the fundus camera on a pupil of a first eye of the subject, such as by focusing/aligning an imaging path of the fundus camera with the black center of the subject's eye. Once focused/aligned, the operator presses the shutter release on the fundus camera, which causes a flash (e.g., a camera flash, an illumination flash) to be fired such that an image and/or photograph of the interior surface of the subject's eye may be captured for subsequent analysis and/or processing. The operator may repeat this procedure to capture an image/photograph of the interior surface of the subject's other eye.

While the external alignment of the fundus imager to the pupil of subject's eye may be externally controlled, the gaze angle orientation of the eye itself is dependent on the subject's cooperation by looking at different fixation targets at the specific time point when the image is taken. Typical fundus imagers may not externally control the gaze angle of the eye. For example, only after the retinal image is taken, an operator of the fundus imager may decide whether the subject cooperated in picking the correct gaze angle and with that generating the correct field of the subject's retina.

The inventors have recognized that there are several technical challenges with conventional fundus imaging. One technical challenge is accurately and/or efficiently aligning an imaging path of a fundus camera with a pupil of a human subject. For example, an operator may manually move the fundus camera such that the imaging path of the fundus camera aligns with the human subject's pupil. Such manual movement may be inaccurate as the operator may not sufficiently align the fundus camera imaging path with the human subject's pupil prior to image capture. Misalignments degrade image quality by inducing unwanted haze in the retinal image or even obstruct part of the illumination or viewing paths altogether. Manual alignment is especially difficult as the patient may constantly move the body's position and/or the angulation of the eye. Additionally, such manual movement of the fundus camera may consume a portion of the subject's eye appointment that may cause other portion(s) of the eye appointment to be truncated and thereby leaving the subject with reduced time to discuss any concerns with their eye health professional (e.g., a medical doctor (e.g., an ophthalmologist), an optometrist, an optician).

Another technical challenge is accurately and/or consistently determining when a human subject is looking in an instructed direction for a particular fundus image capture. For example, the operator may instruct the human subject to look towards a particular direction, such as by looking towards the left (i.e., the human subject looking towards their left), to capture a fundus image of the human subject's optic disc in a particular eye. The operator may view, such as through lens(es) of the fundus camera, whether the subject's eye is looking in the instructed direction. The operator may also be looking at an infrared image of the retina to determine the correct alignment of the eye. The operator may press the shutter release when the operator determines that the subject is looking in the correct direction. However, the subject may inadvertently look elsewhere shortly before the image is taken and the operator may have to recapture the image. With the subject not looking in the instructed direction, the resulting image may be inaccurate such that the intended eye anatomy for capture (e.g., the optic disc) is not present or sufficiently present in the captured image. Additionally, the operator may need to take several images until the subject is looking in the correct direction to capture the desired image. By needing to take several images, the subject may be exposed to extraneous image flashes, which may cause the subject to experience physical discomfort.

Yet another technical challenge is accurately and/or determining when an eye of the human subject is sufficiently opened to facilitate the fundus image capture. For example, a fundus camera may need a pupil to have a width and/or diameter in a range of 3.3 millimeters (mm) to 5 mm to facilitate sufficient fundus capture. As described above, the operator may view the subject's pupil, and/or, more generally, the subject's eye, prior to image capture. The operator may determine that the subject's eye is closed or not sufficiently opened such that a fundus image of the eye may be captured. The operator may instruct the subject to increase the opening of their eye. Similarly, as described above, the subject may involuntarily close (e.g., partially close, fully close) their eye prior to image capture. With the subject's eye being partially or fully closed, the resulting image may be inaccurate such that the intended eye anatomy for capture is not present or sufficiently present in the captured image. The operator may need to take several images until the subject's eye is sufficiently opened to capture the desired image. The several images may thereby expose the subject to extraneous image flashes, which may cause physical discomfort for the subject.

The inventors have developed technology that overcomes the aforementioned technical challenges. In some embodiments, the inventors' technology includes a fundus camera system that effectuates and/or implements gaze angle triggered fundus imaging. For example, the fundus camera system can be configured to align (e.g., automatically align) an imaging path associated with the fundus camera system with one or both pupils of a human subject and, after determining that the human subject is gazing (i.e., looking) in a desired direction, can be configured to capture a fundus image of the human subject. In some embodiments, the fundus camera system may capture a fundus image associated with a single eye of the subject. In some embodiments, the fundus camera system may simultaneously capture fundus images of both eyes of the subject.

In some embodiments, the fundus camera system can include a fundus camera to capture a fundus image of one or both of a human subject's eyes. In some embodiments, the fundus camera can be moveable (e.g., automatically moveable) such that an imaging path of the fundus camera can be aligned (e.g., automatically aligned) with at least one of the human subject's pupils. For example, the fundus camera system can include a stereo camera to generate and/or output a stereo image of the at least one of the human subject's pupils. In some such embodiments, the fundus camera system can perform triangulation to determine a position of the pupil(s) with respect to the fundus camera based on the stereo image. In some such embodiments, the fundus camera system can control the fundus camera to move (e.g., automatically move) in one or more directions (e.g., one or more of an x-, y-, and/or z-direction, or rotations or a combination thereof) in a camera-based coordinate system to align (e.g., exactly align, align within a pre-defined tolerance) the imaging path of the fundus camera with the pupil(s) based on the position of the pupil(s). The alignment tolerance might be implemented dynamically based on the individual subject's pupil size. On larger pupils the pre-defined alignment tolerance might be larger than in smaller pupils. Beneficially, the example fundus camera system overcomes the technical challenge of accurately and/or efficiently aligning the imaging path of the fundus camera with the pupil(s) by tracking position(s) of the subject's pupil(s) and moving (e.g., automatically moving) the fundus camera based on the tracked position(s). Additionally or alternatively, the fundus camera system may include a prism configuration (e.g., a split prism configuration) to split an image of the human subject's pupils into two or more portions for gaze angle triggered fundus imaging as disclosed herein.

In some embodiments, the fundus camera system may determine a gaze angle of at least one of the subject's pupils by analyzing and/or evaluating a stereo image of the at least one of the subject's pupils optionally in combination with using preliminary infrared (IR) fundus images. For example, the fundus camera system may detect and/or determine major and minor axes of a pupil relative to a position of the pupil in a camera-based coordinate system. The example fundus camera system may estimate and/or determine a gaze angle based on the major and minor axes of the imaged pupil. The example fundus camera system may determine a direction in which the subject is gazing (i.e., looking) based on at least one of the position of the pupil (with respect to and/or in relation to the fundus camera) or the gaze angle. In some embodiments, the fundus camera system can execute one or more machine-learning models using the stereo image, or portion(s) thereof, as input(s) to generate output(s), which can include an identification of a gaze angle of pupil(s) detected in the stereo image. In other embodiments the camera might use the location of the retina optical disc in the preliminary IR fundus image as a proxy of the eye's gaze angle. After determining that the subject is looking in an instructed direction, the fundus camera may capture (e.g., automatically capture) the fundus image. Beneficially, the example fundus camera system overcomes the technical challenge of accurately and/or consistently determining when a human subject is looking in an instructed direction for a particular fundus image capture by capturing a fundus image after determining pupil positions and/or determining gaze angle(s) associated with the pupil(s) prior to the fundus image capture.

In some embodiments, the fundus camera system can execute one or more machine-learning models to determine whether a subject's eye is sufficiently opened to effectuate an intended fundus image capture. For example, the fundus camera system can execute machine-learning model(s) using the stereo image, or portion(s) thereof, as input(s) to generate output(s), which can include a determination of whether a subject's eye is opened and/or a degree to which the subject's eye is open (e.g., 0% open, 25% open, 50% open, 75% open, 99% open, etc.) in the stereo image. After determining that one or both subject's eyes are sufficiently opened, the fundus camera may capture (e.g., automatically capture) the fundus image. Beneficially, the example fundus camera system overcomes the technical challenge of accurately and/or determining when at least one eye of the human subject is sufficiently opened to effectuate the fundus image capture by determining, based on the output(s) of the machine-learning model(s), whether the at least one eye is open and/or to what degree the at least one eye is open.

In some embodiments, the fundus camera system can determine that an adequate fundus image is unable to be captured due to a misplacement of a subject's head in the face shape matching goggle interface (e.g., eye goggles) of the fundus camera system. For example, if a subject misplaces their head in the eye goggles and is therefore positioned in an orientation where the fundus camera cannot get an adequate picture of the subject's fundus, then the subject is to be re-adjusted to the proper position with the eye goggles. In some embodiments, if the stereo camera cannot get an adequate picture of one or more of the subject's eyes, detected using the example pupil detection technique described herein, the fundus camera system can provide and/or otherwise output feedback to the subject to cause the subject to re-adjust their head position for improved fundus imaging.

In some embodiments, the feedback is audio feedback, haptic feedback, visual feedback, and/or a combination thereof. The feedback can be used to improve a subject's fit in the face shape matching goggle interface such that the subject's pupils can be detected and/or, more generally, such that the subject's eyes are in an intended position for fundus image capture. By way of example, the fundus camera system can output audio feedback by outputting audio from at least one speaker. In some such embodiments, the audio feedback can include audible requests (e.g., commands, directions, instructions) to the subject to move the subject's head in a specified direction. With the use of audio stereo speakers, additional directionality of instructions for the subject can be encoded by utilizing only the left speaker for directions towards the left while centered stereo can be used for up and down instructions.

In another example, the fundus camera system can output haptic feedback by outputting vibration from at least one haptic actuator to convey to the subject to move their head in a specified direction. In yet another example, the fundus camera system can output visual feedback by projecting an image on at least one display device with visual requests (e.g., natural language text, an icon, a graphic) to the subject to move the subject's head in a specified direction. In yet another example, the fundus camera system can output audio feedback and visual feedback to the subject to cause the subject to move their head in a direction indicated by the audio and visual feedback.

It should be appreciated that techniques described herein may be implemented alone or in any combination. It should also be appreciated that techniques described herein may be used in imaging and/or measuring devices that are not necessarily operated by the subject to image the subject's own eyes. For example, techniques described herein may be used in imaging devices configured for conventional settings such as hospitals and clinics for use that is assisted by one or more clinicians and/or technicians, as embodiments described herein are not so limited.

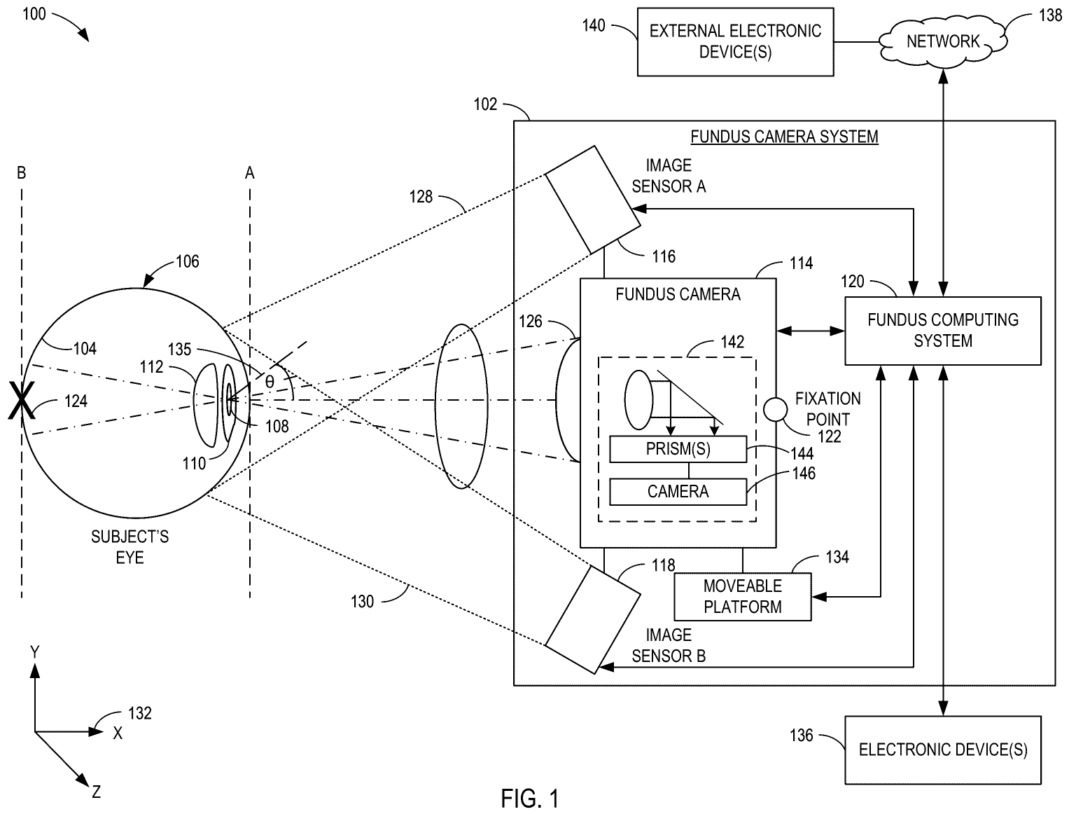

Turning to the figures, the illustrated example of FIG. 1 depicts an example fundus camera environment 100 including an example fundus camera system 102 that can be configured to capture an image of a retina 104 of an eye 106 of a subject through a pupil 108 of the eye 106. Also depicted is an iris 110 and a lens 112 of the eye 106. Other eye anatomical objects, such as the sclera, choroid, and cornea are not shown for enhanced clarity, but nonetheless may be present. Although only one eye 106 is depicted, it should be understood that the fundus camera system 102 can be configured to capture an image of multiple retinas, such as the retina 104 of the eye 106, which is shown, and another retina of a different eye of the subject, which is not shown. For example, the fundus camera system 102 can be configured to capture a first fundus image of the retina 104 and a second fundus image of the other retina substantially simultaneously (e.g., within 10 milliseconds (ms), 50 ms, 100 ms, etc., of each other). The eye 106 of the subject of this example is a human eye of a human subject. Non-limiting examples of human subjects include infants, children, adolescents, adults, and older adults (e.g., adults 65 years of age or older). Alternatively, the eye 106 may be an animal eye of an animal subject. Non-limiting examples of animal subjects include mice, cats, dogs, primates, and horses.

The fundus camera system 102 of the illustrated example includes a fundus camera 114 that can be configured to capture an image, such as a fundus image, of the retina 104. The fundus camera system 102 of this example includes a first image sensor 116 (identified by IMAGE SENSOR A) and a second image sensor 118 (identified by IMAGE SENSOR B). In some embodiments, the first image sensor 116 and/or the second image sensor 118 can be a charge-coupled device (CCD) or a complementary metal oxide semiconductor (CMOS) sensor. Any other type of image sensor is contemplated.

In some embodiments, the first image sensor 116 can be and/or otherwise implement a first camera. In some embodiments, the second image sensor 118 can be and/or otherwise implement a second camera. The first image sensor 116 and the second image sensor 118 of this example can be configured to form a stereo camera. Together, the first image sensor 116 and the second image sensor 118 may implement stereo photography. For example, imaging paths of the first image sensor 116 and the second image sensor 118 may form a triangle such that a position of the pupil 108 may be determined by triangulation associated with the imaging paths.

In some embodiments, the fundus camera system 102 can include and/or implement a three-dimensional (3D) visualization system configured to combine a first image (or photograph), or portion(s) thereof, captured by the first image sensor 116 and a second image (or photograph), or portion(s) thereof, captured by the second image sensor 118 to generate and/or output a 3D image (e.g., a stereo image). For example, the 3D visualization system can be implemented at least by the first image sensor 116 and the second image sensor 118. In some embodiments, the fundus camera system 102 may include and/or use more than two image sensors. Alternatively, the first image sensor 116 and/or the second image sensor 118 may be separate from and/or otherwise not included in the fundus camera system 102.

In the illustrated example, the fundus camera system 102 includes a fundus computing system 120 that can be configured to analyze, evaluate, and/or process outputs from at least one of the fundus camera 114, the first image sensor 116, or the second image sensor 118. The fundus computing system 120 of this example is coupled (e.g., communicatively coupled, electrically coupled, physically coupled) to the fundus camera 114. Alternatively, the fundus computing system 120 may be separate from and/or otherwise not included in the fundus camera system 102.

In some embodiments, the subject may be undergoing an eye exam such that eye drops are administered to the eye cornea (not shown) to cause their pupils to dilate. The subject may be instructed to sit in front of the fundus camera 114 with the subject's face resting in a shape matching goggle interface (not shown) (e.g., goggles or eye goggles) or a chin resting on a chin rest (not shown) and the subject's forehead against a bar or other support structure (not shown). The subject may be instructed to fixate and/or otherwise look at a fixation point 122. The fixation point 122 may be a fixation target implemented by a light projector (not shown). In some embodiments, the fixation point 122 may be a dot or other pattern implemented by light, such as a light emitted by one or more light-emitting diodes (LEDs).

In some embodiments, the fundus camera system 102 may include one or more display devices (not shown) to present image(s) to the subject. For example, the goggles may provide an interface through which the subject may be exposed to the fundus camera 114 and view at least one display device such that the fundus camera system 102 may present image(s) to the subject. Image(s) may include visual feedback to the subject. Examples of visual feedback include graphic(s), icon(s), illustration(s), natural language text, picture(s), and any combination(s) thereof. The visual feedback may be provided to cause the subject to move their head in a specified direction. For example, the visual feedback may be a picture of a human head and a directional arrow indicating a direction in which the subject should turn their head for improved fundus image capture. In another example, the visual feedback may be natural language text of “Please turn your head slightly towards the left” or “Please turn your head slightly towards the right” to instruct the subject to turn their head towards the left or right as directed. In yet another example, the visual feedback may be the picture of the human head and directional arrow and the natural language text described above.

In some embodiments, the fundus camera system 102 may include one or more speakers (not shown) to provide audio feedback to the subject. For example, the audio feedback may be audio output from speaker(s). The audio feedback may be provided to cause the subject to move their head in a specified direction. For example, the audio feedback may be audible natural language of “Please turn your head slightly towards the left” or “Please turn your head slightly towards the right” to instruct the subject to turn their head towards the left or right as directed. In some embodiments, the fundus camera system 102 may provide audio feedback alone, visual feedback alone, or a combination of audio and visual feedback to the subject to cause the subject to move their head in a specified direction.

In some embodiments, the subject may be instructed to gaze towards the fixation point 122 during the eye exam such that a target eye location 124 (identified by an “X” on the retina 104) of the eye 106 can be recorded and/or captured. For example, the fundus camera 114 can be triggered to capture a fundus image of the target eye location 124 by leveraging a first imaging path 126 of the fundus camera 114 that traverses into and out of the pupil 108. The target eye location 124 of this example is the macula of the eye 106. The macula of eye 106 is the round area at the center of the retina 104. Alternatively, any other target eye location is contemplated such as the optic discs (not shown).

In some embodiments, the fundus camera system 102 can align (e.g., automatically align) the first imaging path 126 of the fundus camera 114 with the pupil 108 such that the target eye location 124 can be recorded and/or captured. For example, the first image sensor 116 can capture first image data of the eye 106 along a second imaging path 128 and the second image sensor 118 can capture second image data of the eye 106 along a third imaging path 130. The image sensors 116, 118 can output the first and second image data to the fundus computing system 120. The fundus computing system 120 can render the first and second image data into respective first and second images. The fundus computing system 120 can determine whether the pupil 108 is in zero, one, or both images. For example, the fundus computing system 120 may detect the pupil 108 in the first and/or second images.

In some embodiments, the fundus computing system 120 can determine that the pupil 108 is not detected in at least one of the images and/or is not aligned (e.g., approximately aligned within 1 degree, 2 degrees, 5 degrees, etc., of a major and/or minor axis) in at least one of the images. For example, the fundus computing system 120 can determine a position of the pupil 108, based at least in part on the images, in a camera-based coordinate system 132. For example, the fundus camera system can perform triangulation to determine a position of the pupil by using a pupil image in combination with a preliminary IR fundus image or just use the preliminary IR fundus image alone. Alternatively, the fundus camera system can perform triangulation to determine a position of the pupil by using a single pupil camera in combination with a depth finder to correctly locate the position of the eye. In some embodiments, the position of the pupil 108 can have at least one of an x-coordinate corresponding to a coordinate of an x-axis of the camera-based coordinate system 132, a y-coordinate corresponding to a coordinate of a y-axis of the camera-based coordinate system, or a z-coordinate corresponding to a coordinate of a z-axis of the camera-based coordinate system 132. The camera-based coordinate system 132 of this example defines points relative to the axial center of the fundus camera 114. Alternatively, the fundus camera environment 100 may utilize a calibration pattern-based coordinate system in which points are defined relative to a point in the scene.

In some embodiments, the fundus camera system 102 can determine to output feedback (e.g., audio feedback, visual feedback, and/or a combination thereof) to the subject to move their head in a specified direction when the pupil 108 is not detected in at least one of the images and/or is not aligned in at least one of the images. For example, the fundus computing system 120 can determine that the subject needs to move their head towards the subject's left (or the subject's right) based on the position of the pupil 108 or lack of the pupil's detection in at least one of the images. In such an example, the fundus computing system 120 can cause at least one speaker of the fundus camera system 102 to output audio feedback and/or at least one display device of the fundus camera system 102 to output visual feedback to the subject to adjust their head position.

In some embodiments, the fundus computing system 120 can determine to move the fundus camera 114, the first image sensor 116, and the second image sensor 118 such that the pupil 108 is detected in both images and/or is aligned in both images to be subsequently captured by the image sensors 116, 118. For example, the fundus computing system 120 can determine a correction to a position of the fundus camera 114, the first image sensor 116, and the second image sensor 118 to align the first imaging path 126 with the pupil 108. In some such embodiments, the fundus computing system 120 can generate and/or output control signal(s) representative of command(s) to a moveable platform 134 to move the fundus camera 114, the first image sensor 116, and the second image sensor 118. For example, the fundus camera 114, the first image sensor 116, and the second image sensor 118 of FIG. 1 can be physically coupled together, with at least the fundus camera 114 coupled to the moveable platform 134, such that they move together with movement in the moveable platform 134.

In some embodiments, the movement in the moveable platform 134 implements the correction to the position of the fundus camera 114 (and the corresponding positions of the image sensors 116, 118) to improve the alignment of the first imaging path 126 with the pupil 108. For example, after the movement in the moveable platform 134, the image sensors 116, 118 can capture (e.g., recapture) image data associated with the pupil 108 such that a new stereo image of the pupil 108 can be rendered by the fundus computing system 120. In some such embodiments, the fundus computing system 120 may determine that the pupil 108 is detected in both images of the stereo image.

In some embodiments, the fundus computing system 120, and/or, more generally, the fundus camera system 102, can estimate and/or determine a gaze angle 135 of the eye 106 with respect to the first imaging path 126, and/or, more generally, with the fundus camera 114. For example, the fundus computing system 120 may determine a ratio of a first width of the pupil 108 in the first image captured by the first image sensor 116 and a second width of the pupil 108 in the second image captured by the second image sensor 118. In some embodiments, the fundus computing system 120 can determine the gaze angle 135 based on the ratio. By way of example, a ratio of 1.0 (or approximately 1.0) can represent the eye 106 looking in a straight direction towards the fundus camera 114. In some such embodiments, the fundus computing system 120 can determine that the ratio of 1.0 (or approximately 1.0) corresponds to a gaze angle of 0 degrees (or approximately 0 degrees). By way of another example, the fundus computing system 120 can determine a ratio of 1.5, which can represent the eye 106 looking towards the first image sensor 116. In such an example, the fundus computing system 120 can determine that the ratio of 1.5 corresponds to a gaze angle of +45 degrees. Values for the ratios and gaze angles are examples, and any other value(s) is/are contemplated.

In some embodiments, the fundus camera 114 can capture an image, such as a fundus image, of the target eye location 124 after at least one of (i) a detection of the pupil 108 in both images of the stereo image, (ii) an alignment of the first imaging path 126 with the pupil 108, or a determination and/or verification of the gaze angle 135 of the pupil 108 with respect to the fundus camera 114. Beneficially, the fundus camera 114 may overcome the technical challenges of conventional fundus cameras by capturing the image of the target eye location 124 after determining that the pupil 108 is detected (e.g., the eye 106 is open) and gazing in a direction instructed by the operator of the fundus camera system 102 with reduced and/or otherwise minimized operator intervention. Beneficially, by moving (e.g., automatically moving) the fundus camera 114 to align the first imaging path 126 with the pupil 108 and capturing an image of the target eye location 124 in response to determining that the gaze angle 135 of the eye 106 is a desired gaze angle, the fundus camera 114, and/or, more generally, the fundus camera system 102, can capture the image with improved accuracy and/or efficiency compared to conventional fundus cameras.

In some embodiments, the fundus image can be displayed on a graphical user interface (GUI) implemented by electronic device(s) 136. Non-limiting examples of electronic devices include laptop computers, tablet computers, cellular phones (e.g., smartphones), televisions (e.g., smart televisions), and wearable devices (e.g., headsets, smartwatches, smart glasses, etc.). For example, the fundus computing system 120 can output the fundus image to the electronic device(s) 136 via one or more interfaces. Non-limiting examples of interfaces include BLUETOOTH®, an Ethernet interface, a near-field communication (NFC) interface, a Universal Serial Bus (USB) interface (e.g., USB Type-A, USB Type-B, USB TYPE-C™ or USB-C™, etc.), etc., and/or any combination(s) thereof. Any other type of interface, such as a communication interface, is contemplated. The electronic device(s) 136 can generate and/or launch a GUI to present the fundus image, and/or information associated thereof, to the operator of the fundus camera system 102. Alternatively, the electronic device(s) 136 may be part of the fundus camera system 102.

In some embodiments, operation of the fundus camera system 102 can be enhanced using machine learning (ML), and/or, more generally, artificial intelligence (AI). ML generally refers to the field of deploying computer algorithms (and/or associated hardware) that improve (e.g., automatically improve, iteratively improve) by the use of data in applications (e.g., real-world applications, simulated applications) to generate outputs and through feedback of an evaluation of the outputs to the computer algorithms. Typically, performing ML involves creating a statistical model (or simply a “model”), which is configured to process data to output inferences and/or predictions. Some ML models may be built and/or generated through an iterative process (referred to as “training”) of ingesting data and evaluating outputs of the ML models. Example training of an ML model may be supervised training, which may include instantiating the ML model, providing the ML model with sample data (referred to as “training data”) that may have labels (e.g., metadata, data tags) to describe the sample data, and comparing output(s) of the ML model with the labels to evaluate accuracy of the ML model based on the comparison(s).

In some embodiments, the fundus computing system 120 may implement and/or include one or more ML models, such as deep learning models. For example, the deep learning models may include a convolutional neural network (CNN). Non-limiting examples of a deep learning model include a graph neural network (GNN), a recurrent neural network (RNN), a multi-layer perceptron, an autoencoder, a generative adversarial network (GAN), a CTC-fitted neural network model, and/or any combination(s) thereof. Non-limiting examples of the one or more ML models include a clustering model, a decision tree, a support vector machine (SVM), a Bayesian network, a hidden Markov model, and/or any combination(s) thereof.

In some embodiments, the one or more ML models may be trained (and/or retrained) using training data. For example, the training data may include images of pupils (i.e., pupil images). In some embodiments, the pupil images and/or, more generally, the training data, may be obtained from a network 138. The network 138 of this example may be implemented by any wired and/or wireless network(s) such as one or more cellular networks (e.g., 4G LTE cellular networks, 5G cellular networks, future generation 6G cellular networks, etc.), one or more data buses, one or more local area networks (LANs), one or more optical fiber networks, one or more private networks, one or more public networks, one or more wireless local area networks (WLANs), etc., and/or any combination(s) thereof. For example, the network 138 may be the Internet, but any other type of private and/or public network is contemplated.

In some embodiments, the fundus computing system 120 can train the one or more ML models using the training data. Although the below description is described in connection with the fundus computing system 120, it should be understood that the below description may additionally or alternatively be applicable to external electronic device(s) 140, such as computer server(s), external to the fundus imaging environment 100. For example, the fundus computing system 120 and/or the external electronic device(s) 140 may instantiate, configure, train, and/or execute the one or more ML models.

In some embodiments, the fundus computing system 120 can train the one or more ML models by applying a supervised learning training algorithm using labeled training data, such as pupil images (e.g., labeled pupil images) obtained from the network 138. For example, the pupil images can be labeled with data (e.g., metadata). Non-limiting examples of labels include identifications of eye anatomy (e.g., identifications of pupils, irises, eyelids, corneas, etc.), a detection and/or an identification of a pupil in an image, an identification of whether an eye is open in an image, a degree to which an eye is open (or closed) in an image, a width of a pupil in an image, and a gaze angle of a pupil in an image. In some embodiments, the pupil images may be stereo images, or portion(s) thereof, such as an image captured by a first image sensor of a stereo camera that includes two or more image sensors.

In some embodiments, the one or more ML models are trained on videos of people looking at various targets in various directions, which teaches the one or more ML models how to draw vectors in the direction of the gaze of the subject. In some such embodiments, the one or more ML models may use that training to draw a vector in the direction of the gaze of the current subject in substantially real time and cause a fundus image to be captured when the vector of the gaze of the subject is aligned with the fundus camera 114.

As an example, the fundus computing system 120 may train a deep learning model (e.g., a neural network) by using stochastic gradient descent. As another example, the fundus computing system 120 may train an SVM to identify decision boundaries of the SVM by optimizing a cost function. As an example, the fundus computing system 120 may: (1) generate inputs to the one or more ML models using the pupil images; (2) label the inputs using the labels; and (3) apply a supervised training algorithm to the generated inputs and corresponding labels. Additionally or alternatively, the fundus computing system 120 may train the one or more ML models by applying an unsupervised learning algorithm and/or a semi-supervised learning algorithm to the training data.

In some embodiments, the fundus computing system 120 may train multiple deep learning models, such as multiple neural networks, using the training data. For example, the fundus computing system 120 may train a first neural network using labeled pupil images to generate outputs representing detections of pupils and/or degrees to which eyes are open to expose the pupils. For example, the fundus computing system 120 may train the first neural network to perform concentric ellipse detection (e.g., concentric circle detection) and/or line detection. In some embodiments, the fundus computing system 120 may train separate neural networks to respectively perform concentric ellipse detection and line detection. In some embodiments, the fundus computing system 120 may train a second neural network using the labeled pupil images to generate outputs representing gaze angles associated with the pupils.

In some embodiments, the fundus computing system 120 may retrain the one or more ML models using new and/or revised portion(s) of the training data on a periodic or aperiodic basis. For example, the fundus computing system 120 may be configured to update a previously trained neural network by updating values of one or more parameters of the neural network using new training data. In some embodiments, the fundus computing system 120 may be configured to update the neural network by training a new neural network using a combination of previously obtained training data and new training data.

In some embodiments, the fundus computing system 120 may be configured to update the one or more ML models in response to any one of different types of events. For example, in some embodiments, the fundus computing system 120 may be configured to update the one or more ML models in response to a user command. As an example, the electronic device(s) 136 may provide a GUI via which the user may command performance of a training process. Additionally or alternatively, the external electronic device(s) 140 may provide a GUI via which the user may command performance of a training process. In some embodiments, the fundus computing system 120 may be configured to update the one or more ML models automatically (e.g., not in response to a user command), for example, in response to a software command. As another example, in some embodiments, the fundus computing system 120 may be configured to update the one or more ML models in response to detecting one or more conditions. For example, the fundus computing system 120 may update the one or more ML models in response to detecting expiration of a period of time. As another example, the fundus computing system 120 may update the one or more ML models in response to receiving a threshold amount of new training data (e.g., a threshold amount of new pupil images).

In some embodiments, the fundus computing system 120 may deploy the one or more ML models for inference operations, which include outputting detections of pupils and/or determinations of gaze angles, after the training process. For example, the fundus computing system 120 may execute (e.g., iteratively execute) the one or more ML models using stereo images, or portion(s) thereof, captured by the image sensors 116, 118 as data inputs to generate data outputs. For example, the fundus computing system 120 may execute the first neural network using at least one of a first image of the pupil 108 from the first image sensor 116 or a second image of the pupil 108 from the second image sensor 118 as first data input(s) to generate a first prediction output, which may indicate a detection and/or an identification of the pupil 108 in zero, one, or both images. In some embodiments, the fundus computing system 120 may execute the second neural network using at least one of the first image or the second image as second data inputs to generate a second prediction output, which may indicate an estimate and/or a determination of a gaze angle of the pupil 108.

Additionally or alternatively, the fundus camera system 102 may include a prism configuration 142, which is shown, to implement a 3D visualization system. For example, the 3D visualization system can be implemented at least in part by the prism configuration 142. The prism configuration 142 of this example can be configured to split an image of the target eye location 124 into two halves with an offset between them. When in focus, the halves line up to form a single image. In some embodiments, the split is horizontal while in other embodiments the split is diagonal. Alternatively, the prism configuration 142 can be configured to split the image into at least three portions.

The prism configuration 142 of this example is a split prism configuration including two or more prisms 144. The prisms 144 may output the split image to a camera 146. The fundus computing system 120 of the illustrated example may process the split image in a similar manner to the stereo image generated by the image sensors 116, 118. For example, the fundus computing system 120 may determine the gaze angle 135 based on the split image. In some embodiments, the fundus computing system 120 may execute one or more ML models using the split image as input to generate at least one output representative of at least the gaze angle.

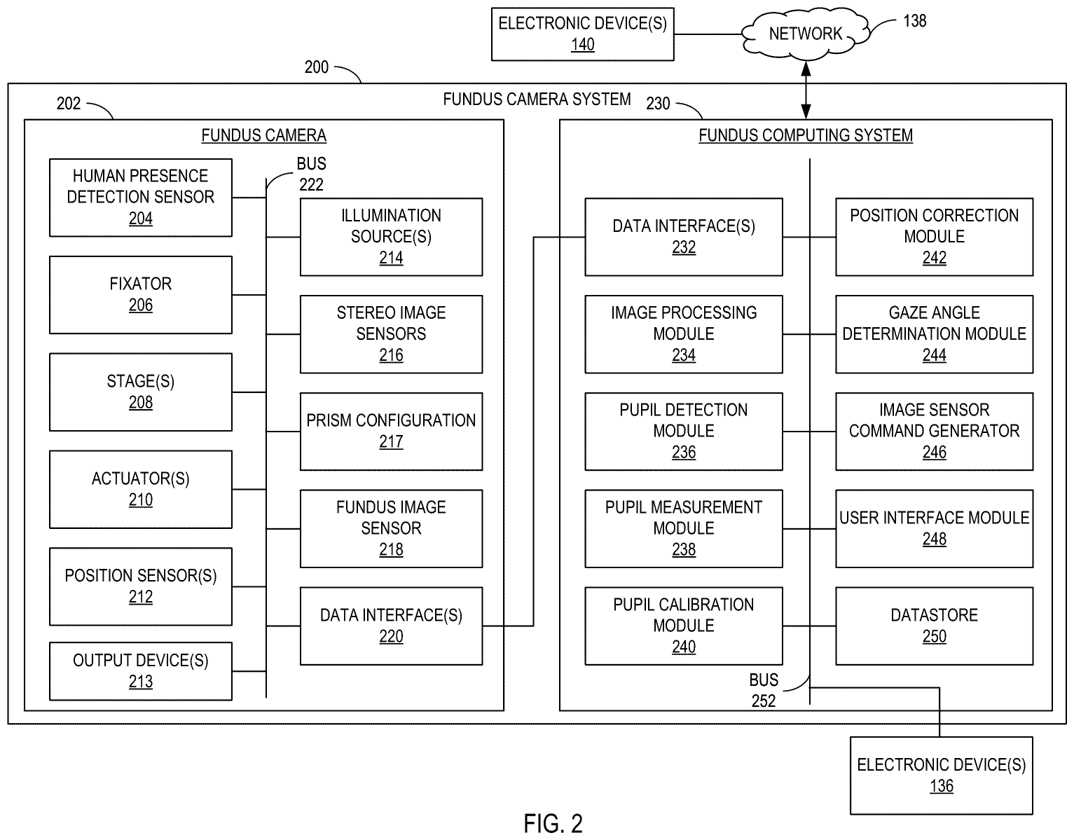

FIG. 2 is a block diagram of an example implementation of a fundus camera system 200, which includes example implementations of a fundus camera 202 and a fundus computing system 230. In some embodiments, the fundus camera system 200 of FIG. 2 can implement and/or correspond to the fundus camera system 102 of FIG. 1. In some embodiments, the fundus camera 202 of FIG. 2 can implement and/or correspond to the fundus camera 114 of FIG. 1. In some embodiments, the fundus computing system 230 of FIG. 2 can implement and/or correspond to the fundus computing system 120 of FIG. 1.

The fundus camera 202 of the illustrated example includes a human presence detection sensor 204, a fixator 206, stage(s) 208, actuator(s) 210, position sensor(s) 212, output device(s) 213, illumination source(s) 214, stereo image sensors 216, a prism configuration 217, a fundus image sensor 218, and first data interface(s) 220. Component(s) of the fundus camera 202 of this example can be in communication with one(s) of each other via a first bus 222. Non-limiting examples of the first bus 222 can be an Inter-Integrated Circuit (I2C) bus, a Peripheral Component Interconnect (PCI) bus, a Peripheral Component Interconnect Express (PCIe) bus, and a Serial Peripheral Interface (SPI) bus. Any other type of bus, such as a computing and/or electrical bus, is contemplated.

The human presence detection sensor 204 of the illustrated example can be configured to detect whether a subject, such as a human subject, is proximate and/or in front of the fundus camera 202. For example, the human presence detection sensor 204 can detect that there is not a human subject in front of the fundus camera 114 and, based on the detection, cause the fundus camera 114 to transition to an idle state (e.g., a standby state, a low power state). In some embodiments, the human presence detection sensor 204 can detect that there is a human subject in front of the fundus camera 114 and, based on the detection, cause the fundus camera 114 to transition from the idle state to an operational state (e.g., an active state, a high power state). Non-limiting examples of the human presence detection sensor 204 are acoustic sensors, infrared (IR) proximity sensors, pressure sensors (e.g., the chin rest and/or the face bar may include and/or embed one or more pressure sensors that can detect whether a human subject is present based on a change in detected pressure), radar sensors, ultrasonic sensors, and vibration sensors.

The fixator 206 of the illustrated example can be configured to generate a fixation point, such as the fixation point 122 of FIG. 1. For example, the fixator 206 can generate a dot or other pattern implemented by light. Non-limiting examples of the fixator 206 include a visible light source and one or more LEDs.

The stage(s) 208 of the illustrated example can be configured to support, hold, and/or move an object, such as the fundus camera 114 of FIG. 1. In some embodiments, the stage(s) 208 can implement and/or correspond to the moveable platform 134 of FIG. 1, or portion(s) thereof. For example, the stage(s) 208 can be one or more boards, planks, or platforms that is/are coupled to the fundus camera 114.

The actuator(s) 210 of the illustrated example can be configured to move and/or translate the stage(s) 208 in one or more directions. In some embodiments, the actuator(s) 210 is/are motors. Any other type of actuator is contemplated. Non-limiting examples of motors include alternating current (AC) brush motors, AC brushless motors, direct current (DC) brush motors, DC brushless motors, stepper motors, servo motors, linear motors, and direct drive motors. Any other type of motor is contemplated. By way of example, the actuator(s) 210 can be operatively coupled to the stage(s) 208, which can be coupled to the fundus camera 114. For example, the actuator(s) 210 can include a plurality of actuators including one or more first actuators, one or more second actuators, and/or one or more third actuators. In some embodiments, the one or more first actuators of the actuator(s) 210 can be operatively coupled to a first stage of the stage(s) 208 such that the one or more first actuators can be actuated and/or otherwise controlled to move the first stage and thereby the fundus camera 114 in a first direction, such as the x-direction of the camera-based coordinate system 132. In some embodiments, the one or more second actuators of the actuator(s) 210 can be operatively coupled to a second stage of the stage(s) 208 such that the one or more second actuators can be actuated and/or otherwise controlled to move the second stage and thereby the fundus camera 114 in a second direction, such as the y-direction of the camera-based coordinate system 132. In some such embodiments, the y-direction is orthogonal to the x-direction. In some embodiments, the one or more third actuators of the actuator(s) 210 can be operatively coupled to a third stage of the stage(s) 208 such that the one or more third actuators can be actuated and/or otherwise controlled to move the third stage and thereby the fundus camera 114 in a third direction, such as the z-direction of the camera-based coordinate system 132. In some such embodiments, the z-direction is orthogonal to the x-direction and the y-direction.

The position sensor(s) 212 of the illustrated example can be configured to detect and/or determine a position of respective one(s) of the stage(s) 208. Non-limiting examples of the position sensor(s) 212 include capacitive displacement sensors, eddy current sensors, Hall effect sensors, inductive sensors, linear variable differential transformers, piezo-electric transducers, position encoders (e.g., absolute encoders, incremental encoders, linear encoders, rotary encoders), potentiometers, proximity sensors, and ultrasonic sensors. By way of example, the position sensor(s) 212 can include one or a plurality of position sensors. In some embodiments, the plurality of position sensors can include a first position sensor configured to determine a position of the first stage of the stage(s) 208, a second position sensor configured to determine a position of the second stage of the stage(s) 208, and/or a third position sensor configured to determine a position of the third stage of the stage(s) 208.

The output device(s) 213 of the illustrated example can be configured to output feedback to the subject. For example, the output device(s) 213 can include one or more speakers, one or more display devices, and/or one or more haptic actuators. In some embodiments, the output device(s) 213 can be one or more speakers that can output audio feedback to the subject. In some embodiments, the output device(s) 213 can be one or more display devices that can present visual feedback to the subject. In some embodiments, the output device(s) 213 can be one or more haptic actuators that can provide tactile and/or vibrotactile feedback to the subject. In some embodiments, the output device(s) 213 can be one or more speakers that can output audio feedback, one or more output devices that can output visual feedback to the subject, and/or one or more haptic actuators that can output haptic, tactile, and/or vibrotactile feedback to the subject.

In some embodiments, one or more haptic actuators may be embedded and/or included in the eye goggles of the fundus camera system 102, the chin rest of the fundus camera system 102, and/or a combination(s) thereof. Non-limiting examples of a haptic actuator include an eccentric rotating mass vibration (ERMV) actuator (e.g., an ERMV motor), a linear resonant actuator (LRA), and a piezoelectric actuator (e.g., a piezo-haptic actuator). Any other haptic actuator is contemplated.

The illumination source(s) 214 of the illustrated example can be configured to emit light momentarily to effectuate capture of an image. For example, the illumination source(s) 214 can be a light source that implements a camera and/or photography flash. Non-limiting examples of the illumination source(s) 214 include transparent glass tubes filled with an inert gas (e.g., xenon gas or other noble gas), a quartz tube filled with an inert gas, and an LED.

The stereo image sensors 216 of the illustrated example can be configured to generate and/or output analog and/or digital data representative of a stereo image. In some embodiments, the stereo image sensors 216 can implement and/or correspond to the first image sensor 116 and/or the second image sensor 118 of FIG. 1. In some embodiments, the stereo image sensors 216 include a plurality of image sensors that can be configured to simulate human binocular vision such that 3D images can be generated. Non-limiting examples of the stereo image sensors 216 include CCD devices and CMOS sensors. Any other type of stereo image sensor is contemplated.

The prism configuration 217 of the illustrated example can be configured to generate and/or output analog and/or digital data representative of a split image. In some embodiments, the prism configuration 217 can implement and/or correspond to the prism configuration 142 of FIG. 1. In some embodiments, the prism configuration 217 includes a plurality of prisms that can be configured to split an image into two or more portions such that 3D images can be generated. A non-limiting example of the prism configuration 217 is a split prism configuration. Any other type of prism configuration is contemplated.

The fundus image sensor 218 of the illustrated example can be configured to generate and/or output a fundus image, such as an image of the target eye location 124 of FIG. 1. In some embodiments, the fundus image sensor 218 can implement and/or correspond to image sensor(s) of the fundus camera 114 of FIG. 1. In some embodiments, the fundus image sensor 218 can include one or a plurality of image sensors. Non-limiting examples of the fundus image sensor 218 include a CCD device and a CMOS sensor. Any other type of fundus image sensor is contemplated.

The first data interface(s) 220 of the illustrated example can be configured to transmit data to and/or receive data from the fundus computing system 230. For example, the first data interface(s) 220 can be a bus. In some embodiments, the first data interface(s) 220 can be one or more communication interfaces.

In the illustrated example of FIG. 2, the fundus camera system 200 includes the fundus computing system 230 to analyze, evaluate, and/or process data, such as image data, captured and/or generated by the fundus camera 202. The fundus computing system 230 of the illustrated example includes second data interface(s) 232, an image processing module 234, a pupil detection module 236, a pupil measurement module 238, a pupil calibration module 240, a position correction module 242, a gaze angle determination module 244, an image sensor command generator 246, a user interface module 248, and a datastore 250. Component(s) of the fundus computing system 230 of this example can be in communication with one(s) of each other via a second bus 252. Non-limiting examples of the second bus 252 can be an I2C bus, a PCI bus, a PCIe bus, and a SPI bus. Any other type of bus, such as a computing and/or electrical bus, is contemplated.

The second data interface(s) 232 of the illustrated example can be configured to transmit data and/or receive data. For example, the second data interface(s) 232 can be a bus. In some embodiments, the second data interface(s) 232 can be one or more communication interfaces.

In some embodiments, the second data interface(s) 232 can be configured to transmit data to and/or receive data from the fundus camera 202 via the first data interface(s) 220. In some embodiments, the second data interface(s) 232 can be configured to transmit data to and/or receive data from the electronic device(s) 136.

In some embodiments, the second data interface(s) 232 can be configured to transmit data to and/or receive data from the external electronic device(s) 140 and/or, more generally, the network 138. For example, the second data interface(s) 232 can implement one or more wireline and/or one or more wireless receivers to facilitate data transfer with the network 138. Non-limiting examples of wireline receivers include Ethernet interfaces and optical interfaces. Any other type of wireline receiver is contemplated. Non-limiting examples of wireless receivers include Wireless Fidelity (Wi-Fi) receivers, cellular modems, and satellite receivers (e.g., beyond-line-of-site (BLOS) satellite receivers, line-of-site (LOS) satellite receivers, etc.).

The image processing module 234 of the illustrated example can be configured to generate and/or render an image based on image data. For example, the image processing module 234 can be configured to receive image data from the stereo image sensors 216 (and/or the prism configuration 217) and render the image data into one or more images, such as a stereo image (and/or split image). In another example, the image processing module 234 can be configured to receive image data from the fundus image sensor 218 and render the image data into one or more images, such as a fundus image.

In some embodiments, the image processing module 234 can process the one or more images. For example, the image processing module 234 can perform glare removal, an illumination correction process, histogram equalization, and/or cropping (e.g., removing, reducing) or masking. For example, the image processing module 234 can crop a portion of a stereo image (and/or split image) that does not include the pupil 108, and/or, more generally, the eye 106. In some embodiments, the image processing module 234 can determine that a stereo image (and/or a split image) includes an image artifact, such as part of the eye goggles of the fundus camera system 102 and crop the part of the image that includes the artifact. Beneficially, the image processing module 234 can process the one or more images as described above to reduce a quantity of computational resources (e.g., processing power, memory bandwidth, storage space) that may be needed to further process the one or more images. For example, by cropping the one or more images to remove extraneous image data, the fundus computing system 230 can process the one or more cropped images with less computational resources with respect to processing uncropped versions of the one or more images.

The pupil detection module 236 of the illustrated example can be configured to detect a presence of a pupil, such as the pupil 108, in an image (e.g., a stereo image, a split image). For example, the pupil detection module 236 can perform one or more image processing techniques and/or execute one or more ML models to detect the pupil 108 in an image. In some embodiments, the pupil detection module 236 can perform at least one of an edge detection technique or a thresholding technique to detect the pupil 108 in the image. In some embodiments, the pupil detection module 236 can provide a stereo image (and/or a split image), or portion(s) thereof, as input(s) to the one or more ML models to generate output(s), which can include indication(s) of whether the pupil 108 is detected in zero, one, or both images of a stereo image (and/or one or more portions of a split image). For example, the pupil detection module 236 can execute the one or more ML models to determine whether the eye 106 is present in the stereo image (and/or the split image), whether a subject is blinking, whether an iris and/or pupil is detected (e.g., by performing concentric ellipse detection), and/or whether an eyelid is detected (e.g., by performing line detection).

In some embodiments in which multiple ML models are used, the pupil detection module 236 can execute the multiple ML models in parallel or sequentially. For example, the pupil detection module 236 can execute a first ML model using a first frame of a stereo image (and/or a first portion of a split image) at a first time and execute a second ML model using a second frame of the stereo image (and/or a second portion of the split image) at the first time to effectuate parallel processing. In some embodiments, the pupil detection module 236 can execute the first ML model at the first time and the second ML model at a second time after the first time to effectuate sequential processing. Alternatively, in some embodiments, the pupil detection module 236 can execute the first ML model at the first time using the first frame and execute the first ML model at the second time using the second frame to reuse the same ML model to effectuate sequential processing.

In some embodiments, the pupil detection module 236 can be configured to determine whether the pupil 108 is sufficiently exposed to the fundus camera 202. For example, the pupil detection module 236 can execute one or more ML models using the stereo image (and/or the split image), or portion(s) thereof, as input(s) to generate output(s), which can include prediction(s). A non-limiting example of the prediction(s) can include whether the pupil 108 is covered by an eyelid of the eye 106 and/or to what degree the pupil 108 is covered. For example, the output(s) of the one or more ML models can indicate that the degree to which the pupil 108 is covered by an eyelid is a particular percentage, such as 0% (e.g., the pupil 108 is entirely exposed), 25%, 50%, 75%, or 100% (e.g., the pupil 108 is entirely covered by the eyelid), or any other percentage.

The pupil measurement module 238 of the illustrated example can be configured to determine a measurement of a dimension associated with the pupil 108 and/or, more generally, the eye 106. Non-limiting examples of the dimension include a depth (or thickness), a height, a width, and a length of the eye 106, or portion(s) thereof, such as the pupil 108. Any other type of dimension and/or measurement(s) thereof is/are contemplated. By way of example, the pupil measurement module 238 can determine a width of the pupil 108 by analyzing and/or evaluating a stereo image (and/or a split image) in which the pupil 108 is detected. For example, the pupil measurement module 238 can determine the width of the pupil 108 by performing an image processing technique on a stereo image (and/or a split image) that includes the pupil 108 and/or by executing one or more ML models that may be trained to output a determination of the width of the pupil 108 based on the stereo image (and/or the split image).

In some embodiments, the pupil measurement module 238 can determine a ratio of measurements associated with the pupil 108 and/or, more generally, the eye 106. For example, the pupil measurement module 238 can determine a ratio of a first width of the pupil 108 depicted in a first image captured by the first image sensor 116 and a second width of the pupil 108 depicted in a second image captured by the second image sensor 118. In some embodiments, the gaze angle determination module 244 as described below can determine a gaze angle of the pupil 108 based on the ratio determined by the pupil measurement module 238.

In some embodiments, the pupil measurement module 238 can determine a position of the pupil 108 in a frame of a stereo image (and/or a portion of a split image). For example, the pupil measurement module 238 can determine and/or detect a position of the pupil 108 relative to major and minor axes of the stereo image frame (and/or the portion of the split image). In some such embodiments, the pupil measurement module 238 can determine a first distance (e.g., a first number of image pixels) that the pupil 108 is from a first axis (e.g., a major axis, an axis of the pupil 108 that is closest to the y-axis of the camera-based coordinate system 132) in a stereo image (and/or a split image portion) and a second distance (e.g., a second number of pixels) that the pupil 108 is from a second axis (e.g., a minor axis, an axis of the pupil 108 that is closest to the x-axis of the camera-based coordinate system 132) in the stereo image (and/or the split image).

The pupil calibration module 240 of the illustrated example can be configured to calibrate component(s) of the fundus camera 202 and/or the fundus computing system 230 based on a measurement associated with the pupil 108 and/or, more generally, the eye 106. For example, the pupil calibration module 240 can adjust one or more tunable parameters associated with the fixator 206, the illumination source(s) 214, the stereo image sensors 216, and/or the fundus image sensor 218 based on a size of the pupil 108, which can include at least one of a depth, a height, a width, or a length of the pupil 108. In some embodiments, the pupil calibration module 240 can adjust one or more tunable parameters associated with the image processing module 234, the pupil detection module 236, the pupil measurement module 238, the position correction module 242, and/or the gaze angle determination module 244 based on the size of the pupil 108.

The position correction module 242 of the illustrated example can be configured to adjust, change, and/or correct a position of the fundus camera 202. In some embodiments, the position correction module 242 can correct one or more coordinates, with respect to the camera-based coordinate system 132, of the fundus camera 114 such that the first imaging path 126 is aligned (or more closely aligned) with the pupil 108. For example, the position correction module 242 can obtain the first and second distances of the pupil 108 (with respect to the first and second axes of the stereo image frame) that may be determined by the pupil measurement module 238. In such an example, the position correction module 242 can receive and/or obtain the coordinates (e.g., an x-coordinate, a y-coordinate, a z-coordinate) of the fundus camera 114 determined by the position sensor(s) 212. Furthering the example, the position correction module 242 can map and/or translate the first and second distances into correction(s) to one(s) of the coordinates. For example, the position correction module 242 can determine to correct an x-coordinate (and/or y-coordinate and/or z-coordinate) of the fundus camera 114 from a first value to a second value of which a difference between the first and second values corresponds to the first and/or second distances. In some such embodiments, the position correction module 242 can determine the correction to the one(s) of the coordinates by using any linear algebra technique such as by using linear transformations and/or rotation matrices. For example, the position correction module 242 can determine the correction by evaluating a relationship between the physical space and the image space associated with the image sensors 116, 118 and the images produced thereby. Any other coordinate correction technique is contemplated.

The gaze angle determination module 244 of the illustrated example can be configured to estimate and/or determine a gaze angle associated with the pupil 108 and/or, more generally, the eye 106. In some embodiments, the gaze angle determination module 244 can determine a gaze angle of the pupil 108 based on the ratio of width measurements determined by the pupil measurement module 238. For example, the gaze angle determination module 244 may determine that the subject is looking in a first direction by determining that a pupil in a first frame of a stereo image (and/or a first half of a split image) is narrower than the pupil in a second frame of the stereo image. In another example, the gaze angle determination module 244 may determine that the subject is looking in a second direction, opposite the first direction, by determining that the pupil in the first frame of the stereo image is wider than the pupil in the second frame of the stereo image (and/or the second half of the split image).

By way of example, the gaze angle determination module 244 can map a ratio of width measurements to a corresponding gaze angle. For example, the gaze angle determination module 244 can map a ratio of 1 (or approximately 1) to a corresponding gaze angle of 0 degrees (or approximately 0 degrees). In another example, the gaze angle determination module 244 can map a ratio of 0.5 to a corresponding gaze angle of −45 degrees, which can indicate the subject is looking towards the subject's right direction. By way of another example, the gaze angle determination module 244 can map a ratio of 1.5 to a corresponding gaze angle of +45 degrees, which can indicate the subject is looking towards the subject's left direction.

In some embodiments, the gaze angle determination module 244 can estimate and/or determine a gaze angle associated with the pupil 108 and/or, more generally, the eye 106, by executing one or more ML models. For example, the gaze angle determination module 244 can execute ML model(s) with a stereo image (and/or a split image), or portion(s) thereof, as input(s) to generate output(s) which can include an indication of the gaze angle and/or, more generally, the gaze angle. In some embodiments, the ML model(s) can be trained using training data including pupil images labeled with respective gaze angles. For example, the ML model(s) can output one or more likelihoods (e.g., probabilities) that a gaze angle of the pupil 108 of FIG. 1 corresponds to the labeled gaze angle in one or more training images.

In some embodiments, the gaze angle determination module 244 can verify the gaze angle based on a comparison of a first gaze angle determined based on the ratio of width measurements and a second gaze angle determined by one or more ML models. For example, the gaze angle determination module 244 can determine that a difference between the first gaze angle and the second gaze angle is less than a threshold (e.g., a difference threshold, a gaze angle difference threshold) and thereby satisfies the threshold. In some embodiments, the gaze angle determination module 244 can determine that the subject is looking in the intended or instructed direction based on the difference satisfying the threshold. By way of another example, the gaze angle determination module 244 can determine that the difference between the first and second gaze angles is greater than the threshold and thereby does not satisfy the threshold. In some such embodiments, the gaze angle determination module 244 can determine that the subject is not looking in the intended or instructed direction based on the difference not satisfying the threshold. In some such embodiments, the gaze angle determination module 244 may cause the user interface module 248 as described below to generate and/or output an alert representing that the subject is not looking in the intended/instructed direction.

The image sensor command generator 246 of the illustrated example can be configured to generate and/or output a command to direct, instruct, trigger, and/or cause the fundus camera 202 to capture an image, such as a fundus image, of the target eye location 124 of FIG. 1. In some embodiments, the image sensor command generator 246 can generate and/or output a command to the illumination source(s) 214 and/or the fundus image sensor 218 via the first and second data interface(s) 220, 232 such that the illumination source(s) 214 creates a flash and the fundus image sensor 218 substantially simultaneously captures an image of the target eye location 124. In some embodiments, the image sensor command generator 246 triggers the flash and capture after determining that at least one of the first imaging path 126 is aligned with the pupil 108, the pupil 108 is sufficiently open, or the pupil 108, and/or, more generally, the eye 106, is gazing in a specified direction. Beneficially, by triggering the flash and capture after such determination(s), the image sensor command generator 246 can capture a fundus image with increased accuracy and efficiency compared to conventional fundus cameras.

In some embodiments, the image sensor command generator 246 can cause the illumination source(s) 214 to create a flash automatically and the fundus image sensor 218 to capture an image automatically and substantially simultaneously. For example, the image sensor command generator 246 can control the illumination source(s) 214 and/or the fundus image sensor 218 without operator input. By way of example, the image sensor command generator 246 can invoke and/or trigger the illumination source(s) 214 in response to determining that at least one of the first imaging path 126 is aligned with the pupil 108, the pupil 108 is sufficiently open, or the pupil 108, and/or, more generally, the eye 106, is gazing in a specified direction. In such an example, the image sensor command generator 246 can invoke and/or trigger the fundus image sensor 218 in response to determining that at least one of the first imaging path 126 is aligned with the pupil 108, the pupil 108 is sufficiently open, or the pupil 108, and/or, more generally, the eye 106, is gazing in a specified direction. Beneficially, in some embodiments, the fundus image can be captured without operator input by invoking and/or triggering the illumination source(s) 214 and the fundus image sensor 218 in response to such determination(s) and/or as soon as such determination(s) are made. Additionally or alternatively, the fundus image sensor 218 may be triggered based on a combination of a pupil image and a preliminary fundus image such as a preliminary infrared image of the fundus.

In some embodiments, the image sensor command generator 246 can be configured to generate and/or output a command to direct, instruct, trigger, and/or cause the stereo image sensors 216 to capture an image, such as a stereo image or portion(s) thereof, of the pupil 108 and/or, more generally, the eye 106 of FIG. 1. By way of example, the image sensor command generator 246 may generate a first command to cause the first image sensor 116 of FIG. 1 to capture first image data associated with the pupil 108 and generate a second command to cause the second image sensor 118 of FIG. 1 to capture second image data associated with the pupil 108. In some embodiments, the image sensor command generator 246 can be configured to generate and/or output a command to direct, instruct, trigger, and/or cause the prism configuration 217 to capture an image, such as a split image or portion(s) thereof, of the pupil 108 and/or, more generally, the eye 106 of FIG. 1.

In some embodiments, the image sensor command generator 246 can be configured to perform opportunistic flashing. For example, the image sensor command generator 246 may determine and/or predict an optimal and/or otherwise improved time at which to trigger image capture by using the pupil tracking performed by and/or a blink status determined by the pupil detection module 236. In some such embodiments, the image sensor command generator 246 may determine to capture an image of the pupil 108 when the pupil 108 is in an expected or desired position by using the pupil tracking performed by the pupil detection module 236. Additionally or alternatively, the image sensor command generator 246 may determine to capture the image of the pupil 108 after determining that the eye 106 has blinked.

The user interface module 248 of the illustrated example can be configured to generate, instantiate, and/or launch a user interface that can be presented to an operator of the fundus camera system 200. In some embodiments, the user interface module 248 can display an image, such as a stereo image (and/or a split image), of the pupil 108 on the user interface. In some embodiments, the user interface module 248 can output feedback, such as a display of an alert and/or outputting of specific instructions to the user and/or patient. Non-limiting examples of the alert include an indication that the subject is closing their eye 106 such that the pupil 108 is covered, the pupil 108 is not sufficiently large to effectuate fundus image capture, the subject is looking in a different direction than the instructed direction (e.g., looking to the subject's left instead of to the subject's right), and the subject is not looking sufficiently towards the instructed direction (e.g., the subject needs to look further to the left or right).

In some embodiments, the user interface module 248 can generate, output, and/or provide feedback indicative of the alert. For example, the user interface module 248 can command, direct, instruct, and/or otherwise control at least one of the output device(s) 213 to provide feedback to a subject and/or an operator. Non-limiting examples of feedback that may be provided include audible feedback, haptic and/or tactile (e.g., vibrotactile) feedback, visual feedback, and/or any combination(s) thereof.

In some embodiments, the user interface module 248 can generate, output, and/or provide audible feedback to a patient and/or an operator. For example, the user interface module 248 can output audio feedback, such as an audible alert, via the output device(s) 213 (e.g., one or more speakers). By way of example, the audible alert can be a human voice based message such as “Please open your eyes wider” after determining that the pupil 108 is covered and/or not sufficiently opened. By way of another example, the audible alert can be a human voice based message such as “Please look to your left” after determining that the gaze angle of the pupil 108 is incorrect and/or the pupil 108 is not detected in at least one image. Any other types of alerts and/or human voice based messages are contemplated.

In some embodiments, the user interface module 248 can generate, output, and/or provide haptic and/or tactile feedback to a patient and/or an operator. For example, the user interface module 248 can control the output device(s) 213 to output a vibration or any other vibrotactile feedback. For example, the user interface module 248 can control the haptic actuator to output the vibration to cause the patient to make an adjustment, such as moving the patient's head towards a particular direction (e.g., vibrating a haptic actuator in a left eye goggle to cause the patient to look more towards the patient's left direction) and/or increasing an eye opening of the eye 106. In such an example, the haptic actuator may output the vibration after a determination that the pupil 108 is not detected in at least one image.

In some embodiments, the user interface module 248 can generate, output, and/or provide visual feedback to an operator. For example, the user interface module 248 can output, using the electronic device(s) 136, a real-time visual (e.g., a substantial real-time visual within 1 second of real-time) for the operator that may show a target position (e.g., a bullseye graphic, a crosshair graphic) and a current alignment of the pupil 108 with respect to the target position. In some embodiments, the real-time visual may include one or more slider bars. Non-limiting examples of slider bars include a slider bar for pupil dimension and a slider bar for blink status.

In some embodiments, the user interface module 248 may output visual feedback to a subject using the fixator 206. For example, the visual feedback may be implemented by a change in a position of a fixation target based on physical tendencies of a patient to look and/or fixate in a particular direction. For example, instead of moving the fundus camera system 102, or portion(s) thereof, to adjust for a gaze of a patient, the user interface module 248 may output visual feedback implemented by a command to the fixator 206 to move the fixation point 122 to a current direction of the eye 106.

In some embodiments, the user interface module 248 may output visual feedback to a subject using the output device(s) 213 (e.g., one or more display devices). For example, the user interface module 248 may generate and/or output graphic(s), icon(s), illustration(s), natural language text, picture(s), and any combination(s) thereof for presentation to the subject. The visual feedback may be provided to cause the subject to move their head in a specified direction. For example, the visual feedback may be a picture displayed on the output device(s) 213 of a human head and a directional arrow indicating a direction in which the subject should turn their head for improved fundus image capture. In another example, the visual feedback may be natural language text of “Please turn your head slightly towards the left” or “Please turn your head slightly towards the right” displayed on the output device(s) 213 to instruct the subject to turn their head towards the left or right as directed. In yet another example, the visual feedback may be the picture of the human head and directional arrow and the natural language text described above displayed on the output device(s) 213.

The datastore 250 of the illustrated example can be configured to record and/or store data. Non-limited examples of recorded/stored data include an image (e.g., a stereo image, a split image, or portion(s) thereof), sequential sequence(s) of image(s), alert(s), notification(s), video, eye and/or pupil measurements, ML model(s), ML training data, and ML labels. Any other type of recorded/stored data is contemplated. For example, the stereo image sensors 216, and/or, more generally, the fundus camera 202, may capture raw video and store the captured raw video in the datastore 250.

In some embodiments, the datastore 250 can be implemented by any technology for storing data. For example, the datastore 250 can be implemented by a volatile memory (e.g., a Synchronous Dynamic Random Access Memory (SDRAM), a Dynamic Random Access Memory (DRAM), a RAMBUS Dynamic Random Access Memory (RDRAM), etc.) and/or a non-volatile memory (e.g., flash memory). The datastore 250 may additionally or alternatively be implemented by one or more double data rate (DDR) memories, such as DDR, DDR2, DDR3, DDR4, mobile DDR (mDDR), etc. The datastore 250 may additionally or alternatively be implemented by one or more mass storage devices such as hard disk drive(s) (HDD(s)), compact disk (CD) drive(s), digital versatile disk (DVD) drive(s), solid-state disk (SSD) drive(s), etc. While in the illustrated example the datastore 250 is illustrated as a single datastore, the datastore 250 may be implemented by any number and/or type(s) of datastore. Furthermore, the data stored in the datastore 250 may be in any data format. Non-limiting examples of data formats include a flat file, binary data, comma delimited data, tab delimited data, and structured query language (SQL) structures.

In some embodiments, the datastore 250 may be implemented by a database system, such as one or more databases. The term “database” as used herein means an organized body of related data, regardless of the manner in which the data or the organized body thereof is represented. For example, the organized body of related data may be in the form of one or more of a table, a log, a map, a grid, a packet, a datagram, a frame, a file, an e-mail, a message, a document, a report, a list or in any other form.

While an example implementation of the fundus camera 202, the fundus computing system 230, and/or, more generally, the fundus camera system 200, is depicted in FIG. 2, other implementations are contemplated. For example, one or more blocks, components, functions, etc., of the fundus camera 202, the fundus computing system 230, and/or, more generally, the fundus camera system 200, may be combined or divided in any other way. The fundus camera 202, the fundus computing system 230, and/or, more generally, the fundus camera system 200 of the illustrated example may be implemented by hardware alone, or by a combination of hardware, software, and/or firmware. For example, the fundus camera 202, the fundus computing system 230, and/or, more generally, the fundus camera system 200, may be implemented by one or more analog or digital circuits (e.g., comparators, operational amplifiers, etc.), one or more hardware-implemented state machines, one or more programmable processors (e.g., central processing units (CPUs), digital signal processors (DSPs), field programmable gate arrays (FPGAs), graphics processing units (GPUs), etc.), one or more network interfaces (e.g., network interface circuitry, network interface cards (NICs), smart NICs, etc.), one or more application specific integrated circuits (ASICs), one or more memories (e.g., non-volatile memory, volatile memory, etc.), one or more mass storage disks or devices (e.g., HDDs, SSD drives, etc.), etc., and/or any combination(s) thereof.

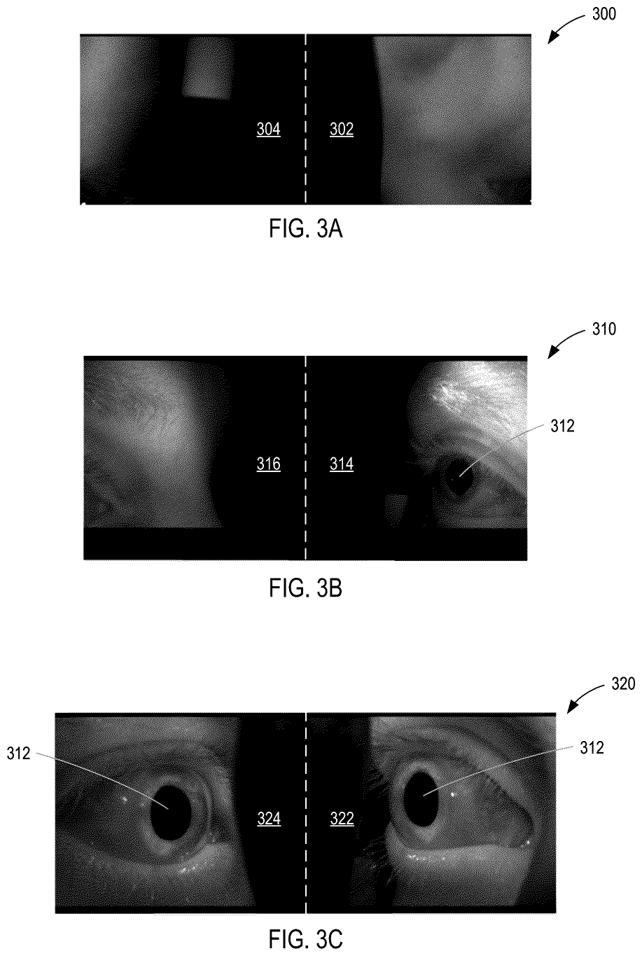

FIG. 3A is a first image 300 of a human subject in which no pupils are detected in frames 302, 304 of the first image 300. The first image 300 of FIG. 3A is a stereo image including a first frame 302 (e.g., a first image frame) and a second frame 304 (e.g., a second image frame). In some embodiments, the first image 300 can be rendered and/or generated by the image processing module 234 of FIG. 2 based on image data of the eye 106 of FIG. 1 captured by the stereo image sensors 216 of FIG. 2. For example, the first frame 302 can be captured by the first image sensor 116 of FIG. 1 and the second frame 304 can be captured by the second image sensor 118 of FIG. 1. In such an example, the image processing module 234 can form the first image 300 based on a combination of the first frame 302 and the second frame 304.

In some embodiments, the pupil detection module 236 of FIG. 2 can determine, based on the first image 300, that no pupil is detected in either frame 302, 304. For example, the pupil detection module 236 can execute ML model(s) using the first image 300, or portion(s) thereof such as the first frame 302 and/or the second frame 304, as input(s) to generate output(s), which can include an indication that the pupil 108 of FIG. 1 (or any other pupil) is not detected in either frame 302, 304.

In some embodiments, the position correction module 242 of FIG. 2 can determine a correction to a position of the fundus camera 114 such that the pupil 108 may be captured in at least one of the frames 302, 304. For example, the position correction module 242 may cause the moveable platform 134 of FIG. 1 to at least be moved away from the subject (e.g., moving along the z-axis of the camera-based coordinate system 132) to capture the pupil 108 in at least one of the first frame 302 or the second frame 304.

FIG. 3B is a second image 310 of the human subject of FIG. 3A in which a pupil 312 is detected in a first frame 314 of the second image 310. In some embodiments, the second image 310 of FIG. 3B can be rendered and/or generated by the image processing module 234 of FIG. 2 based on image data of the eye 106 of FIG. 1 captured by the stereo image sensors 216 of FIG. 2. For example, the first frame 314 can be captured by the first image sensor 116 of FIG. 1 and a second frame 316 of the second image 310 can be captured by the second image sensor 118 of FIG. 1. In such an example, the image processing module 234 can form the second image 310 based on a combination of the first frame 314 and the second frame 316. In some embodiments, the second image 310 of FIG. 3B may be captured after the first image 300 of FIG. 3A. For example, the second image 310 of FIG. 3B can be captured by the image sensors 116, 118 after the fundus camera 114 is moved by the moveable platform 134 of FIG. 1.

In some embodiments, the pupil detection module 236 of FIG. 2 can determine, based on the second image 310, that the pupil 312 is detected in the first frame 314 and not detected in the second frame 316. For example, the pupil detection module 236 can execute ML model(s) using the second image 310, or portion(s) thereof such as the first frame 314 and/or the second frame 316, as input(s) to generate output(s), which can include an indication that the pupil 108 of FIG. 1 is detected in only one of the frames 314, 316.

In some embodiments, the position correction module 242 of FIG. 2 can determine a correction to a position of the fundus camera 114 such that the pupil 108 may be captured in the second frame 316. For example, when the pupil 312 is detected in only one of the frames 314, 316, the position correction module 242 can calculate and/or determine a position correction for the fundus camera 114 in the x- and y-axes of the camera-based coordinate system 132. In such an example, the position correction module 242 may cause the moveable platform 134 of FIG. 1 to be moved in at least one of the x- and y-directions of the camera-based coordinate system 132 such that the image sensors 116, 118 may capture the pupil 108 in the first and second frames 314, 316.

FIG. 3C is a third image 320 of the human subject of FIGS. 3A and/or 3B in which the pupil 312 of FIG. 3B is detected in a first frame 322 and a second frame 324 of the third image 320. In some embodiments, the third image 320 of FIG. 3C can be rendered and/or generated by the image processing module 234 of FIG. 2 based on image data of the eye 106 of FIG. 1 captured by the stereo image sensors 216 of FIG. 2. For example, the first frame 322 can be captured by the first image sensor 116 of FIG. 1 and the second frame 324 can be captured by the second image sensor 118 of FIG. 1. In such an example, the image processing module 234 can form the third image 320 based on a combination of the first frame 322 and the second frame 324. In some embodiments, the third image 320 of FIG. 3C may be captured after the second image 310 of FIG. 3B. For example, the third image 320 of FIG. 3C can be captured by the image sensors 116, 118 after the fundus camera 114 is moved by the moveable platform 134 of FIG. 1.

In some embodiments, the pupil detection module 236 of FIG. 2 can determine, based on the third image 320, that the pupil 312 is detected in the first frame 322 and the second frame 324. For example, the pupil detection module 236 can execute ML model(s) using the third image 320, or portion(s) thereof such as the first frame 322 and/or the second frame 324, as input(s) to generate output(s), which can include an indication that the pupil 108 of FIG. 1 is detected in both frames 322, 324.

In some embodiments, the position correction module 242 of FIG. 2 can determine a correction to a position of the fundus camera 114 such that the pupil 108 may be aligned in the frames 322, 324. For example, when the pupil 312 is detected in both frames 322, 324, the position correction module 242 can calculate and/or determine a position correction for the fundus camera 114 in the z-axis of the camera-based coordinate system 132. In such an example, the position correction module 242 may cause the moveable platform 134 of FIG. 1 to be moved away from the subject in the z-direction of the camera-based coordinate system 132 such that the image sensors 116, 118 may capture the pupil 108 more centrally in the first and second frames 314, 316. Beneficially, in some embodiments, the fundus camera 114 can be controlled to automatically capture a fundus image after and/or in response to determining that the pupil 312 is in both frames 322, 324 and/or is centrally located in both frames 322, 324.

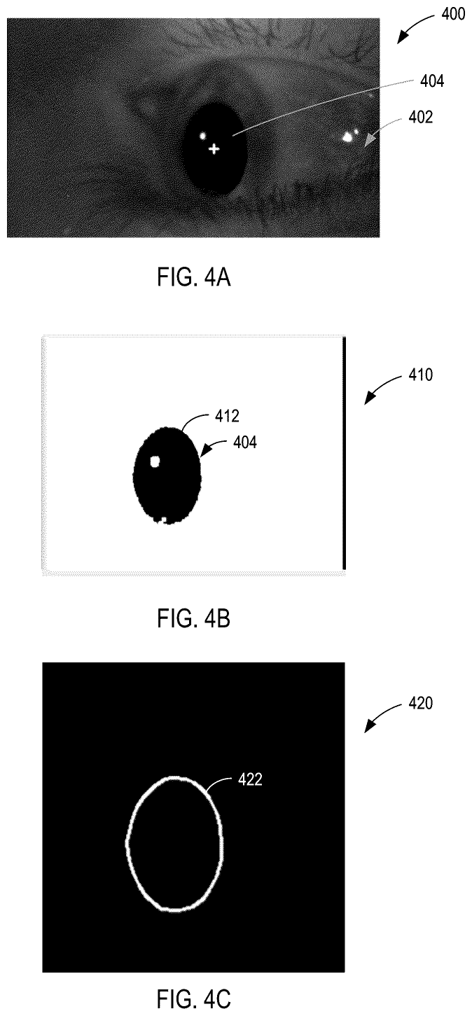

FIG. 4A is an image 400 of an eye 402 of a human subject in which a pupil 404 is detected. The pupil 404 may be distinguished by a very uniform, very dark color. The very uniform, very dark color may be achievable because of the relatively low glare implemented by the fundus camera system 102 of FIG. 1.

In some embodiments, the pupil detection module 236 of FIG. 2 can distinguish the pupil 404 from other parts of the eye 402. For example, the pupil detection module 236 can execute ML model(s) that is/are trained to detect very uniform, very dark colored portions of images and identify the portions as being associated with an eye pupil.

FIG. 4B depicts a digital representation 410 of the pupil 404 of FIG. 4A. In some embodiments, the digital representation 410 can be a pupil candidate region of an image, such as the image 400 of FIG. 4A. In some embodiments, a pupil candidate region can be a portion of an image that may correspond to an eye pupil.

In some embodiments, the pupil detection module 236 can execute one or more ML model(s) to detect the pupil candidate region. For example, the pupil detection module 236 can execute first ML model(s) using the image 400 of FIG. 4A as input(s) to generate output(s), which can include an identification of a portion of the image 400 that includes a very regular elliptical shape 412. For example, the output(s) can indicate an ellipticity of the pupil 404. In such an example, the ellipticity may be representative of a degree to which a shape is elliptical. In this example, the very regular elliptical shape 412 corresponds to the shape of the pupil 404 depicted in FIGS. 4A and 4B. In some embodiments, the first ML model(s) can be trained to perform concentric ellipse detection to identify the very regular elliptical shape 412. Put another way, the pupil detection module 236 can execute the first ML model(s) trained in concentric ellipse detection to identify the very regular elliptical shape 412 and determine that the very regular elliptical shape 412 represents a detection of the pupil 404. Beneficially, even if the pupil 404 is partially occluded by eyelashes, eyelids, or spot glare from an IR illumination source, the first ML model(s) can match partial ellipse curves as being associated with a detection of the pupil 404.

FIG. 4C depicts a digital representation 420 of a border 422 of the pupil 404 of FIGS. 4A and/or 4B. In some embodiments, the pupil detection module 236 can execute one or more ML model(s) to detect a border of an eye pupil. For example, the pupil detection module 236 can execute the first ML model(s) and/or second ML model(s) using the image 400 of FIG. 4A and/or the very regular elliptical shape 412 of FIG. 4B as input(s) to generate output(s), which can include an identification of the border 422 of the pupil 404. In this example, the border 422 corresponds to an outer perimeter of the pupil 404 depicted in FIGS. 4A and 4B. In some embodiments, the first ML model(s) and/or the second ML model(s) can be trained to perform line detection to identify an outer perimeter of an object, such as the border 422. Put another way, the pupil detection module 236 can execute the first ML model(s) and/or the second ML model(s) trained in line detection to identify the border 422 and determine that the border 422 represents a detection of the pupil 404.

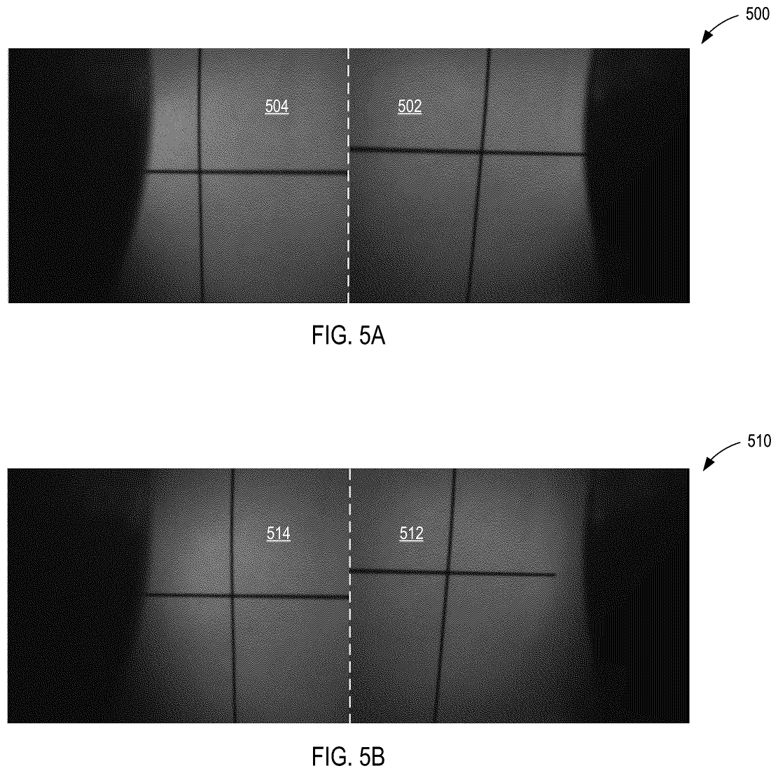

FIG. 5A is a first stereo image 500 that may be captured by the fundus camera system 102 of FIG. 1 using a first set of coordinates. For example, a first frame 502 and a second frame 504 of the first stereo image 500 can be captured by the first and second image sensors 116, 118 of FIG. 1, respectively. In this example, a pupil is not detected in either frame 502, 504.

In some embodiments, in response to the lack of pupil detection, the position correction module 242 of FIG. 2 can determine a change in a position of the fundus camera 114 and thereby a change in respective positions of the image sensors 116, 118 to facilitate pupil detection. For example, any movement of a feature point in 3D space in the stereoscopic view field can be reflected as a translation in the x- and y-coordinates in each of the stereoscopic cameras implemented by the image sensors 116, 118. In some such embodiments, the position correction module 242 can calculate and/or determine a 3-axis correction for any arbitrary offset in 3D space by quantifying a degree of x- and/or y-image translation caused by a given unit movement along each of the 3D axes (e.g., the x-, y-, and z-axes of the camera-based coordinate system 132 of FIG. 1.

FIG. 5B is a second stereo image 510 that may be captured by the fundus camera system 102 of FIG. 1 using a second set of coordinates corrected from the first set of coordinates of FIG. 5A. For example, a first frame 512 and a second frame 514 of the second stereo image 510 can be captured by the first and second image sensors 116, 118 of FIG. 1, respectively, after a change in the position of the fundus camera 114 and the image sensors 116, 118. For example, the second stereo image 510 can represent a difference in crosshair position when the fundus camera 114 and the image sensors 116, 118 are moved a distance (e.g., 5 mm or any other distance) towards a target, such as a human subject's eye, in the z-axis of the camera-based coordinate system 132 of FIG. 1.

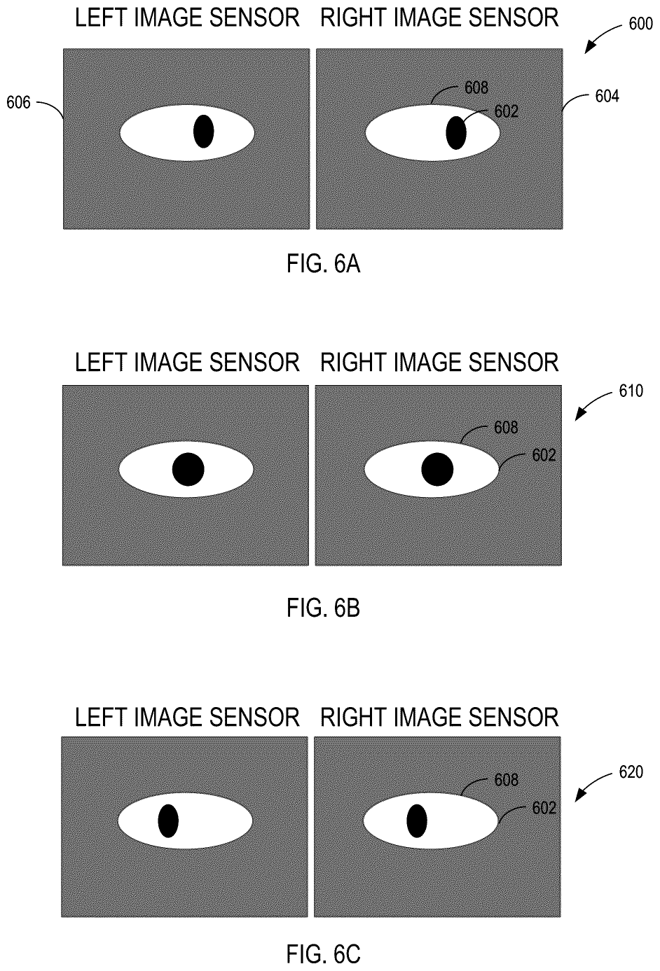

FIG. 6A is an illustration of a first stereo image 600 of a subject's pupil 602 in which the subject is looking in a first direction. In this example, the first stereo image 600 includes a first frame 604 and a second frame 606 in which the pupil 602 is detected in both frames 604, 606. In some embodiments, the first frame 604 can be captured by the first image sensor 116 of FIG. 1 and the second frame 606 can be captured by the second image sensor 118 of FIG. 1.

In the illustrated example, the first direction is a left direction from the perspective of the subject. Put another way, the subject in FIG. 6A is looking in the subject's left direction. For example, an operator of the fundus camera system 102 may instruct the subject to look in the left direction such that an optical disc of the subject's eye 608 can be captured in an image (e.g., a fundus image, an optical disc image).

FIG. 6B is an illustration of a second stereo image 610 of the subject's pupil 602 of FIG. 6A in which the subject is looking in a second direction. In this example, the second direction is a middle direction from the perspective of the subject. Put another way, the subject in FIG. 6B is looking in a straight direction. For example, an operator of the fundus camera system 102 may instruct the subject to look straight towards the fundus camera 114 of FIG. 1 such that a macula of the subject's eye 608 can be captured in an image (e.g., a fundus image, a macula image).

FIG. 6C is an illustration of a third stereo image 620 of the subject's pupil 602 of FIGS. 6A and/or 6B in which the subject is looking in a third direction. In this example, the third direction is a right direction from the perspective of the subject. Put another way, the subject in FIG. 6C is looking in the subject's right direction. For example, an operator of the fundus camera system 102 may instruct the subject to look in the right direction such that a periphery of the subject's eye 608 can be captured in an image (e.g., a fundus image, a periphery image). Although three examples of different images are depicted in FIGS. 6A-6C, any other type of image is contemplated in connection with an eye.

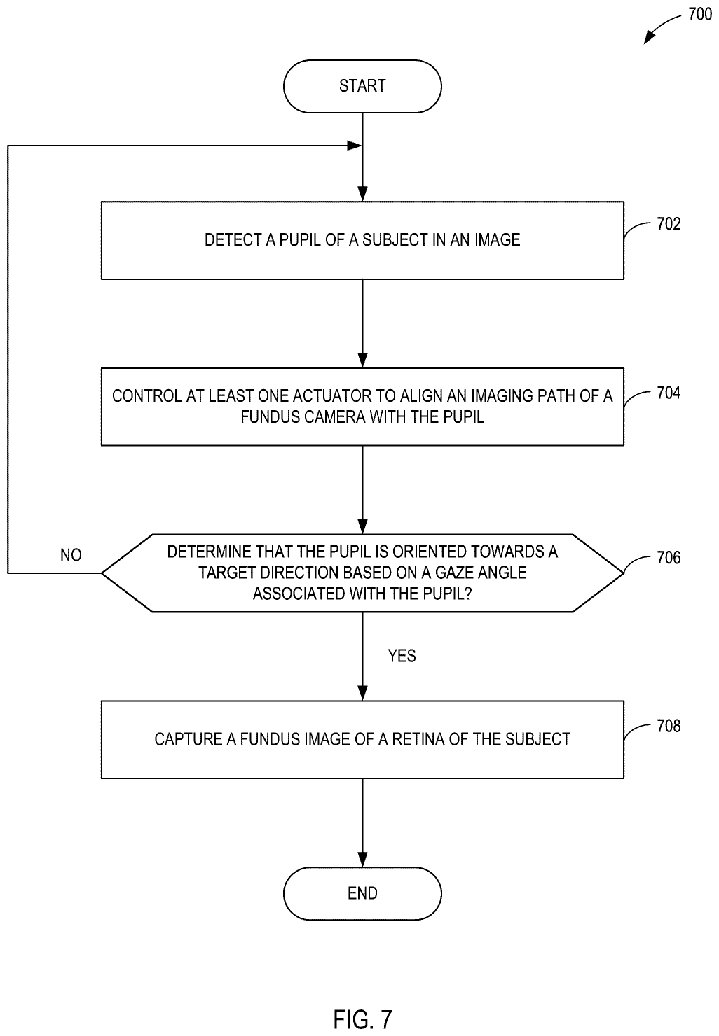

FIGS. 7-13 are flowcharts representative of machine-readable instructions that may be executed by processor circuitry to implement a fundus camera, such as the fundus camera 114, 202 of FIGS. 1 and/or 2, a fundus computing system, such as the fundus computing system 120, 230 of FIGS. 1 and/or 2, and/or, more generally, a fundus camera system, such as the fundus camera system 102, 200 of FIGS. 1 and/or 2. Additionally or alternatively, block(s) of one(s) of the flowcharts of FIGS. 7, 8, 9, 10, 11, 12, and/or 13 may be representative of state(s) of one or more hardware-implemented state machines, algorithm(s) that may be implemented by hardware alone such as an ASIC, etc., and/or any combination(s) thereof. Although some block(s) of the flowcharts of FIGS. 7, 8, 9, 10, 11, 12, and/or 13 is/are described in connection with stereo image(s), additionally or alternatively, the block(s) may be implemented using portion(s) of image(s) from a prism configuration, such as the prism configuration 142 of FIG. 1 and/or the prism configuration 217 of FIG. 2.

FIG. 7 is a flowchart 700 representative of an example process that may be performed to perform gaze angle triggered fundus imaging. In some embodiments, the flowchart 700 is representative of example machine-readable instructions that may be executed by processor circuitry to implement the fundus camera system 102, 200 of FIGS. 1 and/or 2 to capture a fundus image of a retina of a subject based on a gaze angle associated with a pupil.

The flowchart 700 of FIG. 7 begins at block 702, at which the fundus camera system 102, 200 may detect a pupil of a subject in an image. For example, the pupil detection module 236 of FIG. 2 may execute one or more first ML models using the second image 310 of FIG. 3B as input(s) to generate output(s), which may include an indication that the pupil 312 is detected in the first frame 314 but not the second frame 316 of FIG. 3B. In some embodiments, the pupil detection module 236 may perform pupil detection in a split image output from the prism configuration 142 of FIG. 1 and/or the prism configuration 217 of FIG. 2.

At block 704, the fundus camera system 102, 200 may control at least one actuator to align an imaging path of a fundus camera with the pupil. For example, the position correction module 242 of FIG. 2 may determine a correction to a position of the fundus image sensor 218 (and thereby a position of the stereo image sensors 216) to align the first imaging path 126 of FIG. 1 with the pupil 108 of FIG. 1 based on the correction. In such an embodiment, the position correction module 242 may output the correction to the actuator(s) 210 such that the stage(s) 208 may be moved to implement the correction. Put another way, the fundus computing system 230 may control the actuator(s) 210 to align the first imaging path 126 with the pupil 108 after performing pupil detection.

At block 706, the fundus camera system 102, 200 may determine whether the pupil is oriented towards a target direction based on a gaze angle associated with the pupil. For example, the gaze angle determination module 244 of FIG. 2 may execute one or more second ML models (and/or one(s) of the one or more first ML models) using the third image 320 of FIG. 3C as input(s) to generate output(s), which may include an indication of the gaze angle 135 of the pupil 312 in one or both frames 322, 324 of the third image 320. In some embodiments, the gaze angle determination module 244 may determine that the subject is oriented towards a target direction (e.g., to the subject's left, center, or right) based on the gaze angle 135. In some embodiments, the gaze angle determination module 244 may determine that the subject is not oriented towards the target direction based on the gaze angle 135.

If, at block 706, the fundus camera system 102, 200 determines that the pupil is not oriented towards a target direction based on a gaze angle associated with the pupil, the example flowchart 700 of FIG. 7 may be restarted. For example, the stereo image sensors 216 may recapture a stereo image of the pupil 108, and/or, more generally, the eye 106 of FIG. 1.

If, at block 706, the fundus camera system 102, 200 determines that the pupil is oriented towards a target direction based on a gaze angle associated with the pupil, control proceeds to block 708. At block 708, the fundus camera system 102, 200 may capture a fundus image of a retina of the subject. For example, the image sensor command generator 246 of FIG. 2 may generate a command to cause the fundus image sensor 218 to capture an image (e.g., a fundus image) of the retina 104 of FIG. 1. Put another way, the fundus camera 202 may capture a fundus image of the retina 104 of the subject after determining that the pupil 108 is oriented towards a target direction based on the gaze angle 135 associated with the pupil 108. After capturing a fundus image of a retina of the subject at block 708, the example flowchart 700 of FIG. 7 concludes.

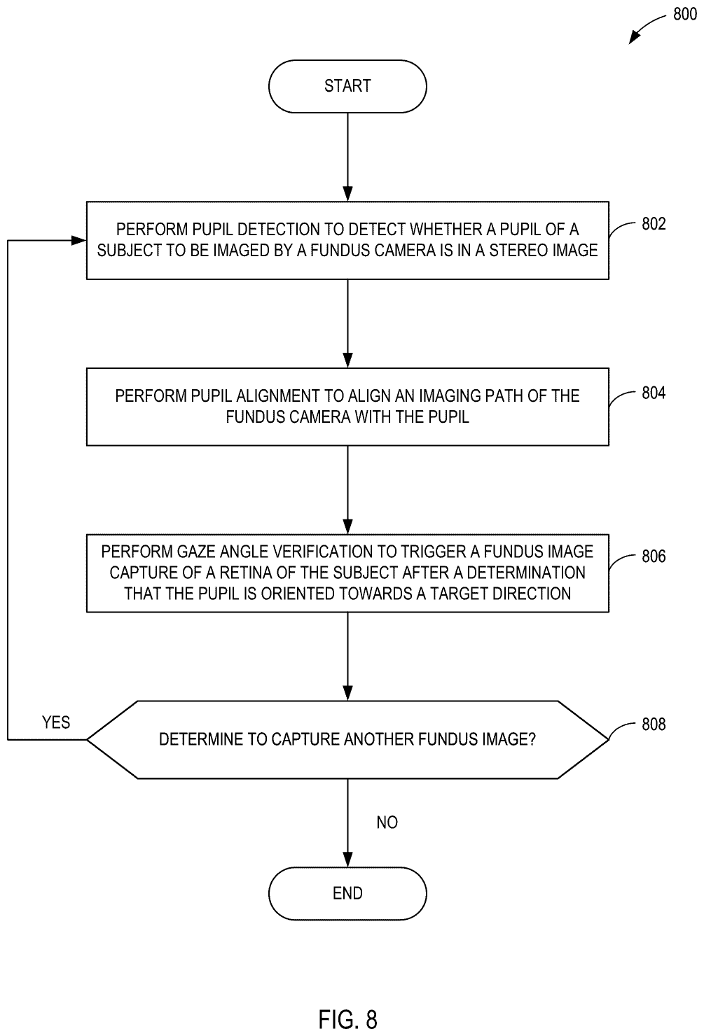

FIG. 8 is a flowchart 800 representative of an example process that may be performed to perform gaze angle triggered fundus imaging. In some embodiments, the flowchart 800 is representative of example machine-readable instructions that may be executed by processor circuitry to implement the fundus camera system 102, 200 of FIGS. 1 and/or 2 to perform gaze angle triggered fundus imaging.

The flowchart 800 of FIG. 8 begins at block 802 at which the fundus camera system 102, 200 of FIG. 1 may perform pupil detection to detect whether a pupil of a subject to be imaged by a fundus camera is in a stereo image. For example, the pupil detection module 236 of FIG. 2 may execute ML model(s) to determine whether the pupil 312 is detected in zero, one, or both frames 322, 324 of the third image 320 of FIG. 3C. In some embodiments, the pupil detection module 236 may determine that the pupil 312 is not detected in the frames 302, 304 of the first image 300 of FIG. 3A based on output(s) of the ML model(s). In some embodiments, the pupil detection module 236 may determine that the pupil 312 of FIG. 3B is detected in the first frame 314 of the second image 310 of FIG. 3B based on output(s) of the ML model(s). In some embodiments, the pupil detection module 236 may determine that the pupil 312 of FIG. 3C is detected in both frames 322, 324 of the third image 320 of FIG. 3C based on output(s) of the ML model(s). An example process that may be performed to implement block 802 is described below in connection with FIG. 8.

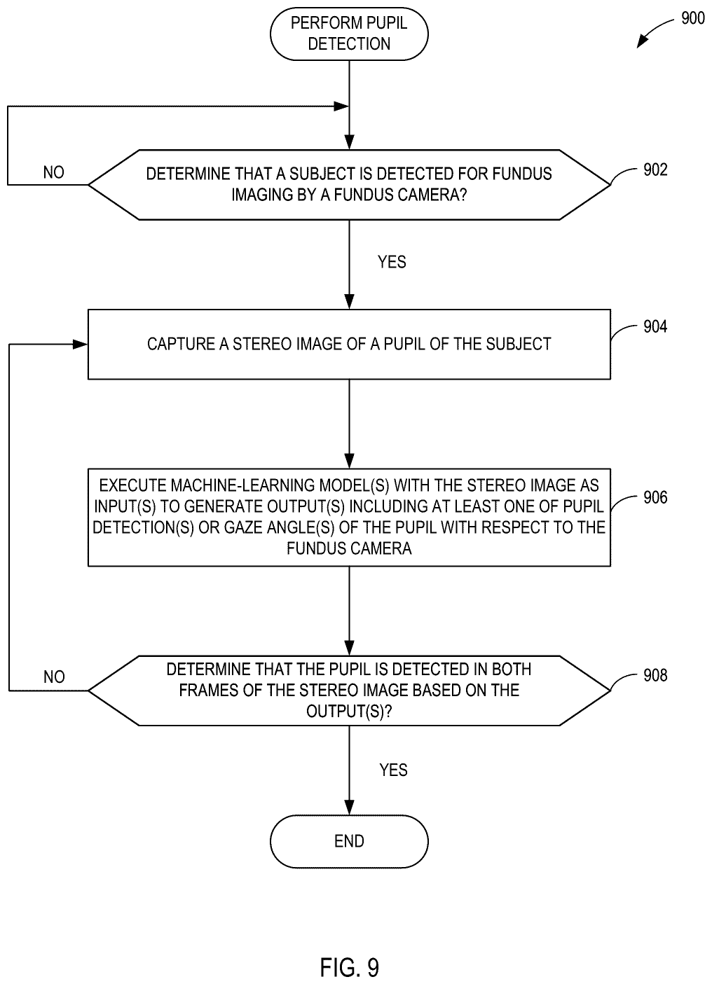

At block 804, the fundus camera system 102, 200 may perform pupil alignment to align an imaging path of the fundus camera with the pupil. For example, the position correction module 242 of FIG. 2 may determine a correction to one or more coordinates of a position of the fundus camera 114 to align the first imaging path 126 of the fundus camera 114 with the pupil 108. In some such embodiments, the position correction module 242 may determine the correction(s) to at least one of detect the pupil 108 in a greater number of frames of a stereo image or more centrally locate the pupil 108 in one or more frames of the stereo image. In some embodiments, the position correction module 242 may cause the moveable platform 134 to move (e.g., automatically move) in accordance with the correction(s). An example process that may be performed to implement block 804 is described below in connection with FIG. 9.

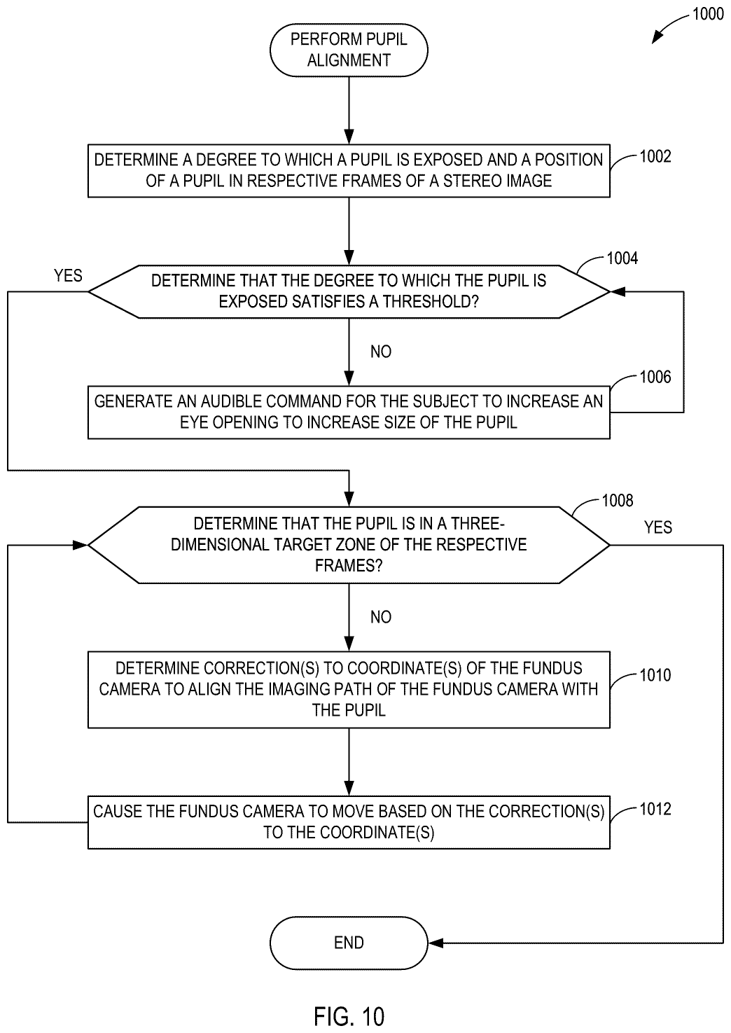

At block 806, the fundus camera system 102, 200 may perform gaze angle verification to trigger a fundus image capture of a retina of the subject after a determination that the pupil is oriented towards a target direction. For example, the gaze angle determination module 244 can determine whether the subject to be fundus imaged is gazing in an instructed direction (e.g., to the subject's left, middle, or right) based on an estimated and/or determined gaze angle. In some embodiments, the gaze angle determination module 244 can determine that the subject is gazing in the instructed direction based on a first gaze angle determined by width measurements associated with the pupil 108, a second gaze angle determined by output(s) of ML model(s), and/or any combination(s) thereof, such as based on a difference between the first and second gaze angles. In some embodiments, after a determination that the subject is gazing in the instructed direction, the imaging sensing command generator 246 of FIG. 2 can command the fundus image sensor 218 of FIG. 2 to trigger capture of a fundus image of the subject's eye. An example process that may be performed to implement block 806 is described below in connection with FIG. 10.

At block 808, the fundus camera system 102, 200 may determine whether to capture another fundus image. For example, the human presence detection sensor 204 of FIG. 2 may detect that the subject is still present in front of the fundus image sensor 218 and thereby determine that the subject may need another fundus capture. By way of another example, the human presence detection sensor 204 may detect that the subject is not present in front of the fundus image sensor 218 and thereby determine that the subject may not need another fundus capture. If, at block 808, the fundus camera system 102, 200 determines to capture another fundus image, control returns to block 802. Otherwise, the example flowchart 800 of FIG. 8 concludes.

FIG. 9 is a flowchart 900 representative of an example process that may be performed to perform pupil detection. In some embodiments, the flowchart 900 is representative of example machine-readable instructions that may be executed by processor circuitry to implement the fundus camera system 102, 200 of FIGS. 1 and/or 2 to perform pupil detection.

The flowchart 900 of FIG. 9 begins at block 902, at which the fundus camera system 102, 200 may determine whether a subject is detected for fundus imaging by a fundus camera. For example, the human presence detection sensor 204 of FIG. 2 may detect that a subject is not present in front of the fundus image sensor 218 and thereby cause the fundus camera 202 to enter and/or remain in an idle state. By way of another example, the human presence detection sensor 204 may detect that a subject is present in front of the fundus image sensor 218 and thereby cause the fundus camera 202 to enter and/or transition to an operational state to facilitate fundus capture.

If, at block 902, the fundus camera system 102, 200 determines that a subject is not detected for fundus imaging by a fundus camera, control waits at block 902, such as to wait until a subject is detected for fundus imaging by the fundus camera. If, at block 902, the fundus camera system 102, 200 determines that a subject is detected for fundus imaging by a fundus camera, control proceeds to block 904.

At block 904, the fundus camera system 102, 200 may capture a stereo image of a pupil of the subject. For example, the stereo image sensors 216 of FIG. 2 may capture images of the pupil 108 of FIG. 1, which may be subsequently processed into a stereo image. In some such embodiments, the image processing module 234 may obtain the images from the stereo image sensors 216 and render the obtained images into a stereo image for subsequent processing by the fundus computing system 230 of FIG. 2.

At block 906, the fundus camera system 102, 200 may execute machine-learning model(s) with the stereo image as input(s) to generate output(s) including at least one of pupil detection(s) or gaze angle(s) of the pupil with respect to the fundus camera. For example, the pupil detection module 236 of FIG. 2 may execute first ML model(s) using the stereo image as input to generate output(s) including indication(s) whether the pupil 108 of FIG. 1 is in zero, one, or two frames of the stereo image. In some embodiments, the pupil detection module 236 may execute the first ML model(s) to determine, if the pupil 108 is detected in a frame, a degree to which the pupil 108 is exposed for fundus imaging (e.g., a degree to which the eye 106 is open or closed). In some embodiments, the gaze angle determination module 244 of FIG. 2 may execute second ML model(s) using the stereo image as input(s) to generate output(s) including a prediction of a gaze angle of the pupil 108 with respect to the camera-based coordinate system 132 of FIG. 1 and/or, more generally, the fundus camera 114 of FIG. 1.

At block 908, the fundus camera system 102, 200 may determine whether the pupil is detected in both frames of the stereo image based on the output(s). For example, the pupil detection module 236 may determine that the pupil 312 is not detected in the frames 302, 304 of the first image 300 of FIG. 3A based on output(s) of the first ML model(s). In some embodiments, the pupil detection module 236 may determine that the pupil 312 of FIG. 3B is detected in the first frame 314 of the second image 310 of FIG. 3B based on output(s) of the first ML model(s). In some embodiments, the pupil detection module 236 may determine that the pupil 312 of FIG. 3C is detected in both frames 322, 324 of the third image 320 of FIG. 3C based on output(s) of the ML model(s). If, at block 908, the fundus camera system 102, 200 determines that the pupil is not detected in both frames of the stereo image based on the output(s), control returns to block 904. Otherwise, the example flowchart 900 of FIG. 9 concludes. In some embodiments, the flowchart 900 of FIG. 9 may return to block 804 of the flowchart 800 of FIG. 8 to perform pupil alignment to align an imaging path of the fundus camera with the pupil.

FIG. 10 is a flowchart 1000 representative of an example process that may be performed to perform pupil alignments. In some embodiments, the flowchart 1000 is representative of example machine-readable instructions that may be executed by processor circuitry to implement the fundus camera system 102, 200 of FIGS. 1 and/or 2 to perform pupil alignment.

The flowchart 1000 of FIG. 10 begins at block 1002, at which the fundus camera system 102, 200 may determine a degree to which a pupil is exposed and a position of a pupil in respective frames of a stereo image. For example, the pupil detection module 236 of FIG. 2 may execute ML model(s) using the second image 310 of FIG. 3B as input(s) to generate output(s), which may include an indication of a degree to which the pupil 312 is exposed to the fundus image sensor 218. In such an example, the pupil detection module 236 may determine that the pupil 312 is 85% exposed such that the eye 106 is open sufficiently to facilitate an adequate fundus image capture. The aforementioned percentage is an example and any other percentage is contemplated. In some embodiments, the pupil measurement module 238 may determine a position of the pupil 602 in the first and second frames 314, 316 of FIG. 3B. For example, the pupil measurement module 238 may determine that the pupil 312 is not aligned in either frame 314, 316 of FIG. 3B.

At block 1004, the fundus camera system 102, 200 may determine whether the degree to which the pupil is exposed satisfies a threshold. For example, the pupil detection module 236 may determine that the degree of 85% is above a threshold (e.g., an exposure threshold, an eye opening threshold) of 70% and thereby satisfies the threshold. In such an example, the degree to which the pupil being exposed satisfying the threshold may be representative of the eye 106 being sufficiently open to facilitate an adequate and/or desired fundus image capture. The aforementioned percentages are examples and any other percentages are contemplated.

If, at block 1004, the fundus camera system 102, 200 determines that the degree to which the pupil is exposed does not satisfy a threshold, control proceeds to block 1006. At block 1006, the fundus camera system 102, 200 may generate an audible command for the subject to increase an eye opening to increase size of the pupil. For example, the pupil detection module 236 may determine that the degree to which the pupil 602 is exposed is 40%, which is below an example threshold of 70% and thereby does not satisfy the threshold. In such an example, the degree to which the pupil being exposed not satisfying the threshold may be representative of the eye 106 not being sufficiently open to facilitate an adequate and/or desired fundus image capture. For example, the eye may be closed or partially closed such that a fundus image of the target eye location 124 of FIG. 1 may not be obtained. In some such embodiments, the user interface module 248 of FIG. 2 may generate and/or output an audible command to cause the subject to open the eye 608 further to increase the degree to which the pupil 602 is exposed. The aforementioned percentages are examples and any other percentages are contemplated.

After generating an audible command at block 1006, control returns to block 1004 to determine, in response to the audible command, whether the degree to which the pupil is exposed satisfies a threshold. For example, the fundus camera system 102, 200 can determine whether an eyelid of the subject reopened or opened sufficiently for fundus image capture. If, at block 1004, the fundus camera system 102, 200 determines that the degree to which the pupil is exposed satisfies a threshold, control proceeds to block 1008.

At block 1008, the fundus camera system 102, 200 may determine whether the pupil is in a three-dimensional target zone of the respective frames. For example, the pupil measurement module 238 may determine whether the pupil 612 in the frames 604, 606 of FIG. 3A have a position in a 3D target zone of the frames 604, 606. In such an example, the pupil measurement module 238 may determine that the 3D target zone is a central or centrally located portion of the frames 604, 606. A 3D target zone may be representative of a portion of a stereo image frame in which a pupil may be detected to effectuate a sufficient fundus capture. In some such embodiments, the pupil measurement module 238 may determine that the pupil 612 in the frames 604, 606 have a position in the central portion of the frames 604, 606 and are therefore in the 3D target zone of the frames 604, 606.

If, at block 1008, the fundus camera system 102, 200 determines that the pupil is in a three-dimensional target zone of the respective frames, the example flowchart 1000 of FIG. 10 concludes. In some embodiments, the flowchart 1000 of FIG. 10 may return to block 806 of the flowchart 800 of FIG. 8 to perform gaze angle verification.

If, at block 1008, the fundus camera system 102, 200 determines that the pupil is not in a three-dimensional target zone of the respective frames, control proceeds to block 1010. At block 1010, the fundus camera system 102, 200 may determine correction(s) to coordinate(s) of the fundus camera to align the imaging path of the fundus camera with the pupil. For example, the position correction module 242 of FIG. 2 may determine a correction to one or more coordinates of the fundus camera 114 to align (e.g., more closely align, substantially align within 1 degree, 2 degrees, 5 degrees, etc.) the first imaging path 126 with the pupil 108.

At block 1012, the fundus camera system 102, 200 may cause the fundus camera to move based on the correction(s) to the coordinate(s). For example, the position correction module 242 may output the correction(s) to the actuator(s) 210 to move the stage(s) 208 such that the fundus image sensor 218 is moved in one or more directions of the camera-based coordinate system 132. After the causing of the fundus camera to move at block 1012, control returns to block 1008 to determine, in response to the moving of the fundus camera, whether the pupil is in a three-dimensional target zone of the respective frames.

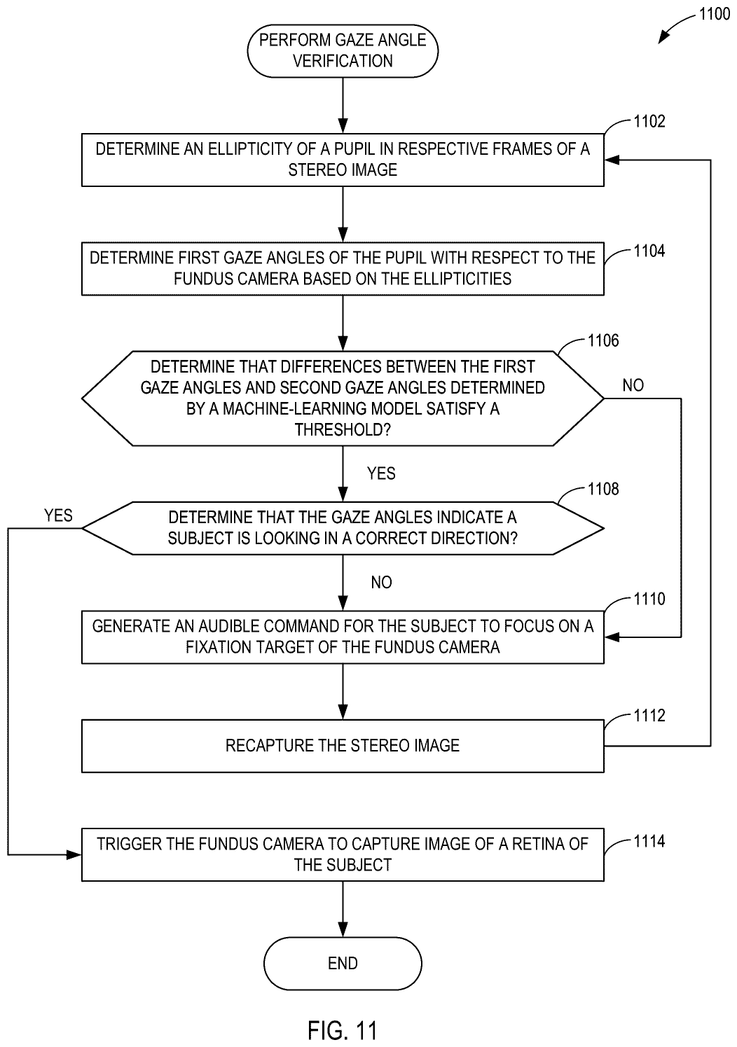

FIG. 11 is a flowchart 1100 representative of an example process that may be performed to perform gaze angle verification. In some embodiments, the flowchart 1100 is representative of example machine-readable instructions that may be executed by processor circuitry to implement the fundus camera system 102, 200 of FIGS. 1 and/or 2 to perform gaze angle verification.

The flowchart 1100 of FIG. 11 begins at block 1102, at which the fundus camera system 102, 200 may determine an ellipticity of a pupil in respective frames of a stereo image. For example, the pupil detection module 236 of FIG. 2 may execute ML model(s) using the image 400 of FIG. 4A as input(s) to generate output(s), which can include a determination of an ellipticity of the pupil 404 of FIG. 4A. In some such embodiments, the pupil detection module 236 may identify the very regular elliptical shape 412 of FIG. 4B based on the ellipticity.

At block 1104, the fundus camera system 102, 200 may determine first gaze angles of the pupil with respect to the fundus camera based on the ellipticities. For example, the gaze angle determination module 244 of FIG. 2 may determine the gaze angle 135 of FIG. 1 as a first gaze angle based on the ellipticity of the pupil 108 in one or more frames of a stereo image.

At block 1106, the fundus camera system 102, 200 may determine whether differences between the first gaze angles and second gaze angles determined by a machine-learning model satisfy a threshold. For example, the gaze angle determination module 244 may determine a second gaze angle based on an output from a ML model trained to estimate and/or predict gaze angles based on images of eye pupils. In some embodiments, the gaze angle determination module 244 may verify an accuracy of the first gaze angle by determining that a difference between the first and second gaze angles is less than a threshold and the difference thereby satisfies the threshold. Alternatively, the gaze angle determination module 244 may determine that the first gaze angle is incorrect and may warrant a recapture of the stereo image to reverify the gaze angle 135 based on a determination that the difference between the first and second gaze angles is greater than a threshold (and the difference thereby does not satisfy the threshold). Beneficially, the gaze angle determination module 244 may determine the gaze angle 135 based on eye geometry and verify the determination with ML techniques as described herein to achieve increased accuracy of gaze angle determination.

If, at block 1106, the fundus camera system 102, 200 determines that differences between the first gaze angles and second gaze angles determined by a machine-learning model do not satisfy a threshold, control proceeds to block 1110.

At block 1110, the fundus camera system 102, 200 generates an audible command for the subject to focus on a fixation target of the fundus camera. For example, the user interface module 248 of FIG. 2 may generate and/or output an audible command (e.g., an audible instruction, an audible directive) to the subject to look towards the fixation point 122 to ensure that the subject is looking in the intended or instructed direction.

At block 1112, the fundus camera system 102, 200 recaptures the stereo image. For example, in response to the audible command, the image sensor command generator 246 of FIG. 2 may output a command to the stereo image sensors 216 to capture another stereo image of the pupil 108. After recapturing the stereo image, control returns to block 1102 to determine (e.g., redetermine) an ellipticity of a pupil in respective frames of a stereo image recaptured at block 1112.

If, at block 1106, the fundus camera system 102, 200 determines that differences between the first gaze angles and second gaze angles determined by a machine-learning model satisfy a threshold, control proceeds to block 1108. At block 1108, the fundus camera system 102, 200 may determine whether the gaze angles indicate a subject is looking in a correct direction. For example, the gaze angle determination module 244 may determine that at least one of the first gaze angle or the second gaze angle indicates that the subject is looking in a direction instructed by the operator of the fundus camera system 102 and/or the fundus camera system 102 (e.g., via an output of the user interface module 248).

If, at block 1108, the fundus camera system 102, 200 determines that the gaze angles do not indicate a subject is looking in a correct direction, control proceeds to block 1110. Otherwise, control proceeds to block 1114.

At block 1114, the fundus camera system 102, 200 may trigger the fundus camera to capture image of a retina of the subject. For example, image sensor command generator 246 may generate a command to cause the fundus image sensor 218 to capture a fundus image of the target eye location 124 of FIG. 1. After triggering the fundus camera at block 1014, the example flowchart 1100 of FIG. 11 concludes. In some embodiments, the flowchart 1100 of FIG. 11 may return to block 808 of the flowchart 800 of FIG. 8 to determine whether to capture another fundus image.

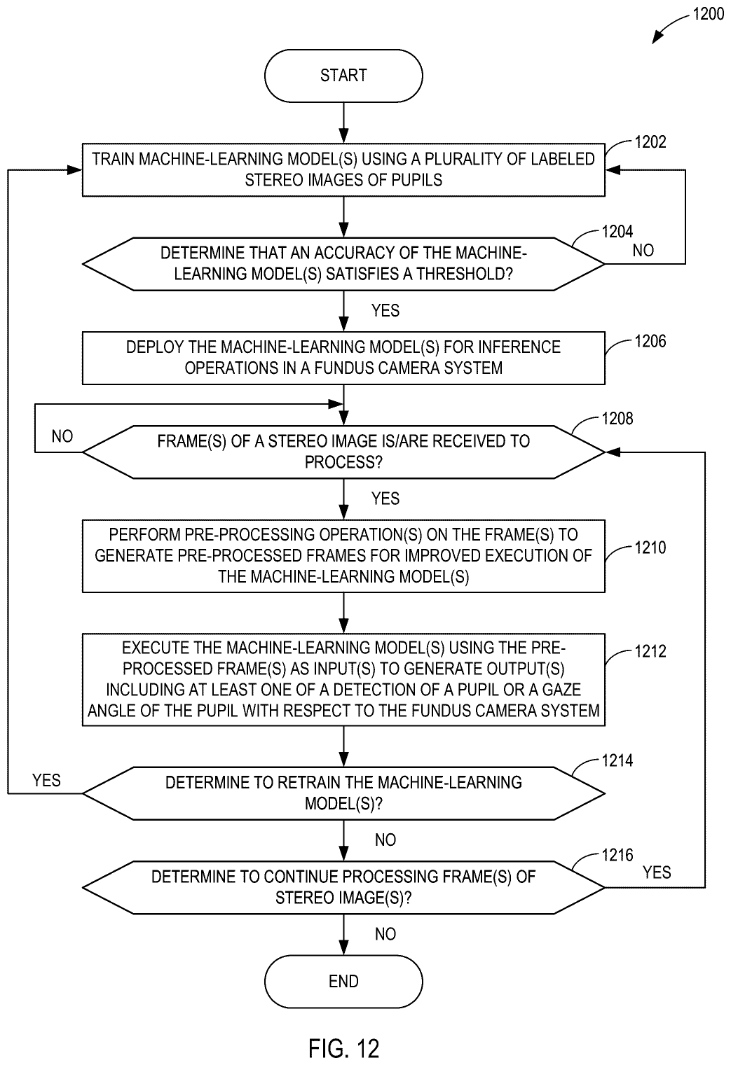

FIG. 12 is a flowchart 1200 representative of an example process that may be performed to train and execute a machine-learning model to generate output(s) including at least one of a detection of a pupil or a gaze angle of the pupil with respect to a fundus camera. In some embodiments, the flowchart 1200 may be representative of example machine-readable instructions that may be executed by processor circuitry to implement the fundus camera system 102, 200 of FIGS. 1 and/or 2 to train and execute a machine-learning model to generate output(s) including at least one of a detection of a pupil or a gaze angle of the pupil with respect to a fundus camera.

The flowchart 1200 of FIG. 12 begins at block 1202, at which the fundus camera system 102, 200 may train machine-learning model(s) using a plurality of labeled stereo images of pupils. For example, the pupil detection module 236 may train first ML model(s) using a plurality of labeled stereo images of pupils for pupil detection. In some embodiments, the gaze angle determination module 244 may train second ML model(s) using the plurality of labeled stereo images of pupils for gaze angle determination and/or verification.

At block 1204, the fundus camera system 102, 200 may determine whether an accuracy of the machine-learning model(s) satisfies a threshold. For example, the pupil detection module 236 may determine whether an accuracy of the first ML model(s) satisfies a threshold (e.g., an accuracy threshold). In some embodiments, the gaze angle determination module 244 may determine whether an accuracy of the second ML model(s) satisfies the threshold (or a different threshold).

If, at block 1204, the fundus camera system 102, 200 determines that an accuracy of the machine-learning model(s) does not satisfy a threshold, control returns to block 1202. For example, the pupil detection module 236 may retrain (or continue to train) the first ML model(s) and/or the gaze angle determination module 244 may retrain (or continue to train) the second ML model(s).

If, at block 1204, the fundus camera system 102, 200 determines that an accuracy of the machine-learning model(s) satisfies a threshold, control proceeds to block 1206. At block 1206, the fundus camera system 102, 200 may deploy the machine-learning model(s) for inference operations in a fundus camera system. For example, the pupil detection module 236 may compile the first ML model(s) into executable construct(s) (e.g., executable file(s), configuration image(s), machine-readable instructions, etc.) that may be instantiated and/or executed to carry out inference operations in the fundus computing system 230 and/or, more generally, the fundus camera system 200. In some embodiments, the gaze angle determination module 244 may compile the second ML model(s) into executable construct(s) (e.g., executable file(s), configuration image(s), machine-readable instructions, etc.) that may be instantiated and/or executed to carry out inference operations in the fundus computing system 230 and/or, more generally, the fundus camera system 200.

At block 1208, the fundus camera system 102, 200 may determine whether frame(s) of a stereo image is/are received to process. For example, the image processing module 234 of FIG. 2 may determine whether one or more frames of a stereo image are received from the fundus camera 202 via the second data interface(s) 232 of FIG. 2.

If, at block 1208, the fundus camera system 102, 200 determines that frame(s) of a stereo image is/are not received to process, control waits at block 1208, such as to wait until frame(s) of a stereo image is/are received to process. If, at block 1208, the fundus camera system 102, 200 determines that frame(s) of a stereo image is/are received to process, control proceeds to block 1210.

At block 1210, the fundus camera system 102, 200 may perform pre-processing operation(s) on the frame(s) to generate pre-processed frames for improved execution of the machine-learning model(s). For example, the image processing module 234 of FIG. 2 may perform one or more pre-processing operations on the received frame(s), which may include glare removal, an illumination correction process, histogram equalization, and/or cropping (e.g., removing, reducing) or masking. By way of example, cropping portion(s) of a frame may reduce a number of pixels of the frame. Beneficially, the first ML model(s) may generate output(s) with reduced latency compared to processing an uncropped version of the frame that has a greater number of pixels. Beneficially, pre-processing the frame, such as by cropping the frame, the first ML model(s) may operate with improved efficiency by generating output(s) with reduced latency.

At block 1212, the fundus camera system 102, 200 may execute the machine-learning model(s) using the pre-processed frame(s) as input(s) to generate output(s) including at least one of a detection of a pupil or a gaze angle of the pupil with respect to the fundus camera system. For example, the pupil detection module 236 may execute the first ML model(s) using the cropped frame as input(s) to generate output(s), which may include a detection of a pupil in the cropped frame. In some embodiments, the gaze angle determination module 244 may execute the second ML model(s) using the cropped frame as input(s) to generate output(s), which may include a gaze angle of the pupil with respect to the fundus image sensor 218 and/or, more generally, the fundus camera 202.

At block 1214, the fundus camera system 102, 200 may determine whether to retrain the machine-learning model(s). For example, the pupil detection module 236 may determine to retrain the first ML model(s) and/or the gaze angle determination module 244 may determine to retrain the second ML model(s). In some such embodiments, the determination(s) to retrain may be based on a determination that a period of time has elapsed (e.g., a determination to periodically retrain the ML model(s) and/or a determination that a threshold quantity of new training data is available.

If, at block 1214, the fundus camera system 102, 200 determines to retrain the machine-learning model(s), control returns to block 1202. Otherwise, control proceeds to block 1216. At block 1216, the fundus camera system 102, 200 determines whether to continue processing frame(s) of stereo image(s). For example, the image processing module 234 2 may determine whether to continue processing one or more frames of a stereo image received from the fundus camera 202 via the second data interface(s) 232.

If, at block 1216, the fundus camera system 102, 200 determines to continue processing frame(s) of stereo image(s), control returns to block 1208. Otherwise, the example flowchart 1200 of FIG. 12 concludes.

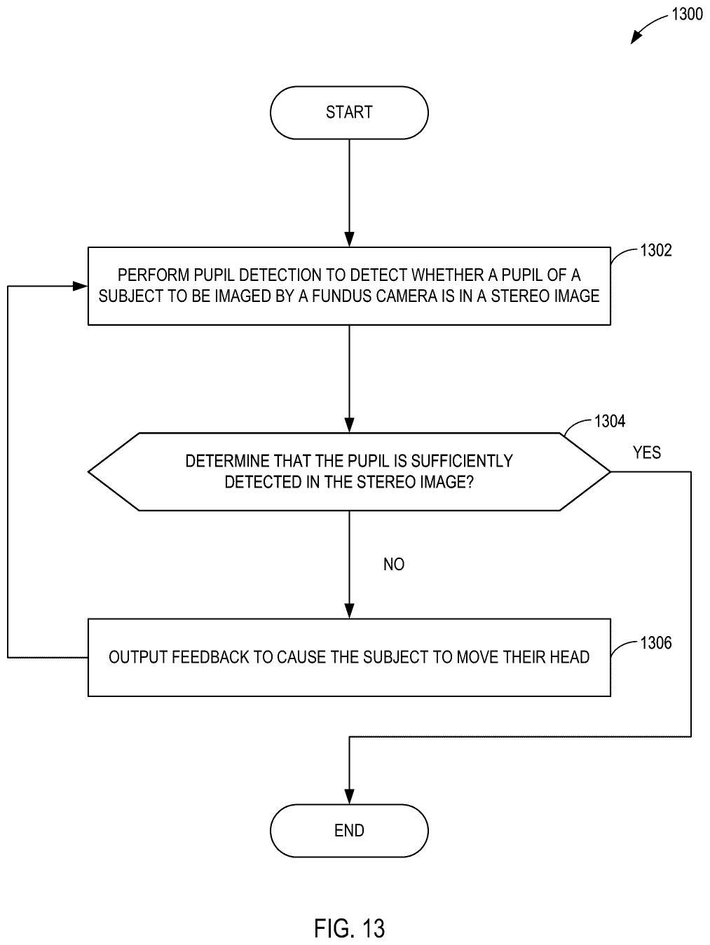

FIG. 13 is a flowchart 1300 representative of an example process that may be performed to output feedback to a subject to improve pupil detection. In some embodiments, the flowchart 1300 may be representative of example machine-readable instructions that may be executed by processor circuitry to implement the fundus camera system 102, 200 of FIGS. 1 and/or 2 to output feedback to a subject to improve pupil detection.

The flowchart 1300 of FIG. 13 begins at block 1302, at which the fundus camera system 102, 200 performs pupil detection to detect whether a pupil of a subject to be imaged by a fundus camera is in a stereo image. For example, the pupil detection module 236 of FIG. 2 may execute ML model(s) to determine whether the pupil 312 is detected in zero, one, or both frames 322, 324 of the third image 320 of FIG. 3C. In some embodiments, the pupil detection module 236 may determine that the pupil 312 is not detected in the frames 302, 304 of the first image 300 of FIG. 3A based on output(s) of the ML model(s). In some embodiments, the pupil detection module 236 may determine that the pupil 312 of FIG. 3B is detected in the first frame 314 of the second image 310 of FIG. 3B based on output(s) of the ML model(s). In some embodiments, the pupil detection module 236 may determine that the pupil 312 of FIG. 3C is detected in both frames 322, 324 of the third image 320 of FIG. 3C based on output(s) of the ML model(s). An example process that may be performed to implement block 802 is described above in connection with FIG. 8.

At block 1304, the fundus camera system 102, 200 determines whether the pupil is sufficiently detected in the stereo image. For example, the pupil detection module 236 can determine that the pupil 312 is detected in zero, one, or both frames 322, 324 of the third image 320 of FIG. 3C. In another example, the pupil detection module 236 can determine that an entirety of the pupil 312 is not detected in one or both frames 322, 324.

If, at block 1304, the fundus camera system 102, 200 determines that the pupil is sufficiently detected in the stereo image, the example flowchart 1300 of FIG. 13 concludes. For example, the fundus camera system 102, 200 can determine that an entirety of the pupil 312 is detected in both frames 322, 324.

If, at block 1304, the fundus camera system 102, 200 determines that the pupil is not sufficiently detected in the stereo image, control proceeds to block 1306. For example, the fundus camera system 102, 200 can determine that only a portion of the pupil 312 is detected in one or both frames 322, 324.

At block 1306, the fundus camera system 102, 200 outputs feedback to cause the subject to move their head. For example, the user interface module 248 of FIG. 2 can control the output device(s) 213 to output at least one of audio feedback, haptic feedback, or visual feedback to cause the subject to move their head in a desired direction for improved fundus imaging such that the pupil 312 is sufficiently detected in the stereo image. After feedback is output to cause the subject to move their head, control returns to block 1302 to determine whether the feedback produced the desired effect by reperforming pupil detection.

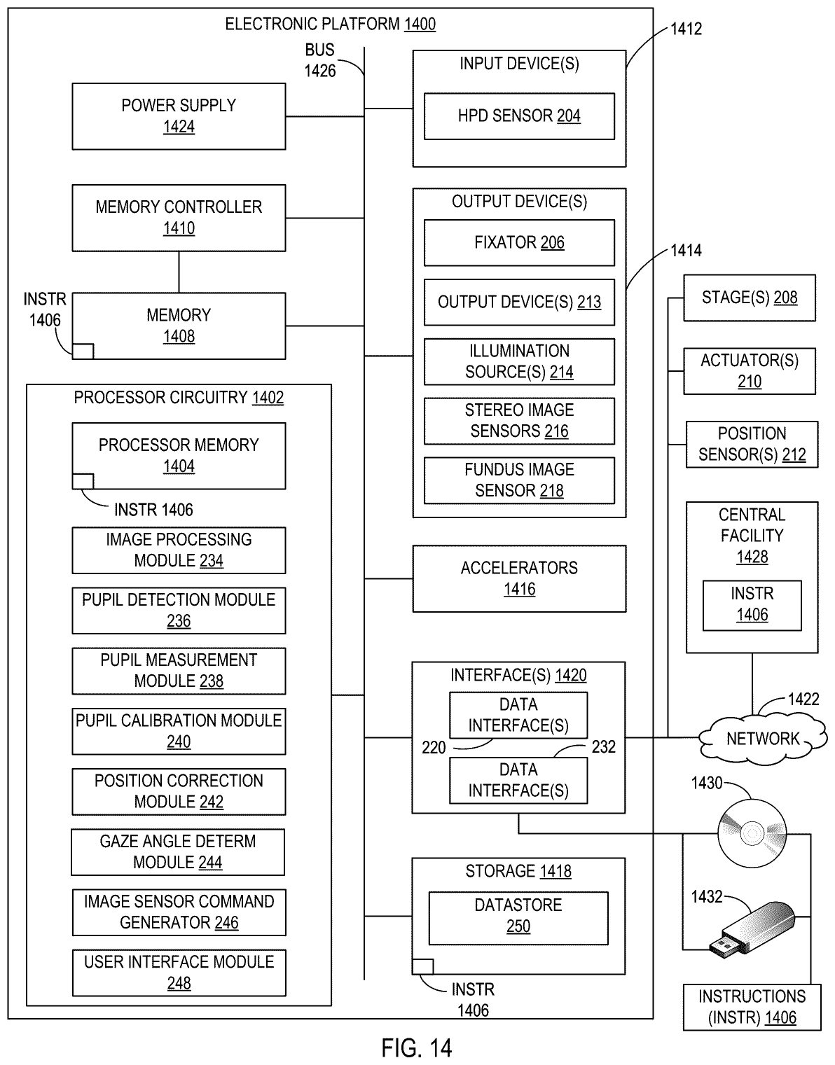

FIG. 14 is an example implementation of an electronic platform 1400 structured to execute the machine-readable instructions of FIGS. 7, 8, 9, 10, 11, 12, and/or 13 to implement a fundus camera system, such as the fundus camera system 102 of FIG. 1 and/or the fundus camera system 200 of FIG. 2. It should be appreciated that FIG. 14 is intended neither to be a description of necessary components for an electronic and/or computing device to operate as a fundus camera system, in accordance with the techniques described herein, nor a comprehensive depiction. The electronic platform 1400 of this example may be an electronic device, such as an industrial computer, a server (e.g., a computer server, a blade server, a rack-mounted server, etc.), a workstation, or any other type of computing and/or electronic device.

The electronic platform 1400 of the illustrated example includes processor circuitry 1402, which may be implemented by one or more programmable processors, one or more hardware-implemented state machines, one or more ASICs, etc., and/or any combination(s) thereof. For example, the one or more programmable processors may include one or more CPUs, one or more DSPs, one or more FPGAs, one or more GPUs, etc., and/or any combination(s) thereof. The processor circuitry 1402 includes processor memory 1404, which may be volatile memory, such as random-access memory (RAM) of any type. The processor circuitry 1402 of this example implements the image processing module 234, the pupil detection module 236, the pupil measurement module 238, the pupil calibration module 240, the position correction module 242, the gaze angle determination module 244 (identified by GAZE ANGLE DETERM MODULE), the image sensor command generator 246, and the user interface module 248 of FIG. 2.

The processor circuitry 1402 may execute machine-readable instructions 1406 (identified by INSTRUCTIONS), which are stored in the processor memory 1404, to implement at least one of the image processing module 234, the pupil detection module 236, the pupil measurement module 238, the pupil calibration module 240, the position correction module 242, the gaze angle determination module 244, the image sensor command generator 246, or the user interface module 248 of FIG. 2. The machine-readable instructions 1406 may include data representative of computer-executable and/or machine-executable instructions implementing techniques that operate according to the techniques described herein. For example, the machine-readable instructions 1406 may include data (e.g., code, embedded software (e.g., firmware), software, etc.) representative of the flowcharts of FIGS. 7, 8, 9, 10, 11, 12, and/or 13, or portion(s) thereof.

The electronic platform 1400 includes memory 1408, which may include the instructions 1406. The memory 1408 of this example may be controlled by a memory controller 1410. For example, the memory controller 1410 may control reads, writes, and/or, more generally, access(es) to the memory 1408 by other component(s) of the electronic platform 1400. The memory 1408 of this example may be implemented by volatile memory, non-volatile memory, etc., and/or any combination(s) thereof. For example, the volatile memory may include static random-access memory (SRAM), dynamic random-access memory (DRAM), cache memory (e.g., Level 1 (L1) cache memory, Level 2 (L2) cache memory, Level 3 (L3) cache memory, etc.), etc., and/or any combination(s) thereof. In some examples, the non-volatile memory may include Flash memory, electrically erasable programmable read-only memory (EEPROM), magnetoresistive random-access memory (MRAM), ferroelectric random-access memory (FeRAM, F-RAM, or FRAM), etc., and/or any combination(s) thereof.

The electronic platform 1400 includes input device(s) 1412 to enable data and/or commands to be entered into the processor circuitry 1402. For example, the input device(s) 1412 may include an audio sensor or any other type of sensor, a camera (e.g., a still camera, a video camera, etc.), a keyboard, a microphone, a mouse, a touchscreen, a voice recognition system, etc., and/or any combination(s) thereof. In this example, the input device(s) 1412 implement the human presence detection sensor 204 (identified by HPD SENSOR) of FIG. 2.

The electronic platform 1400 includes output device(s) 1414 to convey, display, and/or present information to a user (e.g., a human user, a machine user, etc.). For example, the output device(s) 1414 may include one or more display devices, speakers, etc. The one or more display devices may include an augmented reality (AR) and/or virtual reality (VR) display, a liquid crystal display (LCD), a light-emitting diode (LED) display, an organic light-emitting diode (OLED) display, a quantum dot (QLED) display, a thin-film transistor (TFT) LCD, a touchscreen, etc., and/or any combination(s) thereof. The output device(s) 1414 can be used, among other things, to generate, launch, and/or present a user interface. For example, the user interface may be generated and/or implemented by the output device(s) 1414 for visual presentation of output and speakers or other sound generating devices for audible presentation of output. In this example, the output device(s) 1414 implement the fixator 206, the output device(s) 213, the illumination source(s) 214, the stereo image sensors 216, and the fundus image sensor 218 of FIG. 2. Additionally or alternatively, the user interface module 248 may be implemented by the output device(s) 1414.

The electronic platform 1400 includes accelerators 1416, which are hardware devices to which the processor circuitry 1402 may offload compute tasks to accelerate their processing. For example, the accelerators 1416 may include artificial intelligence/machine-learning (AI/ML) processors, ASICs, FPGAs, GPUs, neural network (NN) processors, systems-on-chip (SoCs), vision processing units (VPUs), etc., and/or any combination(s) thereof. In some examples, one or more of the image processing module 234, the pupil detection module 236, the pupil measurement module 238, the pupil calibration module 240, the position correction module 242, the gaze angle determination module 244, the image sensor command generator 246, and/or the user interface module 248 may be implemented by one(s) of the accelerators 1416 instead of the processor circuitry 1402. In some examples, the image processing module 234, the pupil detection module 236, the pupil measurement module 238, the pupil calibration module 240, the position correction module 242, the gaze angle determination module 244, the image sensor command generator 246, and/or the user interface module 248 may be executed concurrently (e.g., in parallel, substantially in parallel, etc.) by the processor circuitry 1402 and the accelerators 1416. For example, the processor circuitry 1402 and one(s) of the accelerators 1416 may execute in parallel function(s) corresponding to the pupil detection module 236.

The electronic platform 1400 includes storage 1418 to record and/or control access to data, such as the machine-readable instructions 1406. In this example, the storage 1418 may implement the datastore 250 of FIG. 2. The storage 1418 may be implemented by one or more mass storage disks or devices, such as HDDs, SSDs, etc., and/or any combination(s) thereof.

The electronic platform 1400 includes interface(s) 1420 to effectuate exchange of data with external devices (e.g., computing and/or electronic devices of any kind) via a network 1422. In this example, the interface(s) 1420 may implement the first data interface(s) 220 and the second data interface(s) 232 of FIG. 2. The interface(s) 1420 of the illustrated example may be implemented by an interface device, such as network interface circuitry (e.g., a NIC, a smart NIC, etc.), a gateway, a router, a switch, etc., and/or any combination(s) thereof. The interface(s) 1420 may implement any type of communication interface, such as BLUETOOTH®, a cellular telephone system (e.g., a 4G LTE interface, a 5G interface, a future generation 6G interface, etc.), an Ethernet interface, a near-field communication (NFC) interface, an optical disc interface (e.g., a Blu-ray disc drive, a Compact Disk (CD) drive, a Digital Versatile Disk (DVD) drive, etc.), an optical fiber interface, a satellite interface (e.g., a BLOS satellite interface, a LOS satellite interface, etc.), a Universal Serial Bus (USB) interface (e.g., USB Type-A, USB Type-B, USB TYPE-C™ or USB-C™, etc.), etc., and/or any combination(s) thereof. Additionally or alternatively, the interface(s) 1420 may implement any other type of interface such as an I2C interface, a PCI interface, a PCIe interface, a SPI interface, and/or the like. For example, the interface(s) 1420 may facilitate communication with the stage(s) 208, the actuator(s) 210, and/or the position sensor(s) 212 via any one(s) of the aforementioned interfaces.

The electronic platform 1400 includes a power supply 1424 to store energy and provide power to components of the electronic platform 1400. The power supply 1424 may be implemented by a power converter, such as an alternating current-to-direct-current (AC/DC) power converter, a direct current-to-direct current (DC/DC) power converter, etc., and/or any combination(s) thereof. For example, the power supply 1424 may be powered by an external power source, such as an alternating current (AC) power source (e.g., an electrical grid), a direct current (DC) power source (e.g., a battery, a battery backup system, etc.), etc., and the power supply 1424 may convert the AC input or the DC input into a suitable voltage for use by the electronic platform 1400. In some examples, the power supply 1424 may be a limited duration power source, such as a battery (e.g., a rechargeable battery such as a lithium-ion battery).

Component(s) of the electronic platform 1400 may be in communication with one(s) of each other via a bus 1426. For example, the bus 1426 may be any type of computing and/or electrical bus, such as an I2C bus, a PCI bus, a PCIe bus, a SPI bus, and/or the like. In some embodiments, the bus 1426 of FIG. 14 can implement and/or correspond to the first bus 222 and/or the second bus 252 of FIG. 2.

The network 1422 may be implemented by any wired and/or wireless network(s) such as one or more cellular networks (e.g., 4G LTE cellular networks, 5G cellular networks, future generation 6G cellular networks, etc.), one or more data buses, one or more local area networks (LANs), one or more optical fiber networks, one or more private networks, one or more public networks, one or more wireless local area networks (WLANs), etc., and/or any combination(s) thereof. For example, the network 1422 may be the Internet, but any other type of private and/or public network is contemplated.

The network 1422 of the illustrated example facilitates communication between the interface(s) 1420 and a central facility 1428. The central facility 1428 in this example may be an entity associated with one or more servers, such as one or more physical hardware servers and/or virtualizations of the one or more physical hardware servers. For example, the central facility 1428 may be implemented by a public cloud provider, a private cloud provider, etc., and/or any combination(s) thereof. In this example, the central facility 1428 may compile, generate, update, etc., the machine-readable instructions 1406 and store the machine-readable instructions 1406 for access (e.g., download) via the network 1422. For example, the electronic platform 1400 may transmit a request, via the interface(s) 1420, to the central facility 1428 for the machine-readable instructions 1406 and receive the machine-readable instructions 1406 from the central facility 1428 via the network 1422 in response to the request.

Additionally or alternatively, the interface(s) 1420 may receive the machine-readable instructions 1406 via non-transitory machine-readable storage media, such as an optical disc 1430 (e.g., a Blu-ray disc, a CD, a DVD, etc.) or any other type of removable non-transitory machine-readable storage media such as a USB drive 1432. For example, the optical disc 1430 and/or the USB drive 1432 may store the machine-readable instructions 1406 thereon and provide the machine-readable instructions 1406 to the electronic platform 1400 via the interface(s) 1420.

Techniques operating according to the principles described herein may be implemented in any suitable manner. The processing and decision blocks of the flowcharts above represent steps and acts that may be included in algorithms that carry out these various processes. Algorithms derived from these processes may be implemented as software integrated with and directing the operation of one or more single- or multi-purpose processors, may be implemented as functionally equivalent circuits such as a DSP circuit or an ASIC, or may be implemented in any other suitable manner. It should be appreciated that the flowcharts included herein do not depict the syntax or operation of any particular circuit or of any particular programming language or type of programming language. Rather, the flowcharts illustrate the functional information one skilled in the art may use to fabricate circuits or to implement computer software algorithms to perform the processing of a particular apparatus carrying out the types of techniques described herein. For example, the flowcharts, or portion(s) thereof, may be implemented by hardware alone (e.g., one or more analog or digital circuits, one or more hardware-implemented state machines, etc., and/or any combination(s) thereof) that is configured or structured to carry out the various processes of the flowcharts. In some examples, the flowcharts, or portion(s) thereof, may be implemented by machine-executable instructions (e.g., machine-readable instructions, computer-readable instructions, computer-executable instructions, etc.) that, when executed by one or more single- or multi-purpose processors, carry out the various processes of the flowcharts. It should also be appreciated that, unless otherwise indicated herein, the particular sequence of steps and/or acts described in each flowchart is merely illustrative of the algorithms that may be implemented and can be varied in implementations and embodiments of the principles described herein.

Accordingly, in some embodiments, the techniques described herein may be embodied in machine-executable instructions implemented as software, including as application software, system software, firmware, middleware, embedded code, or any other suitable type of computer code. Such machine-executable instructions may be generated, written, etc., using any of a number of suitable programming languages and/or programming or scripting tools, and also may be compiled as executable machine language code or intermediate code that is executed on a framework, virtual machine, or container.

When techniques described herein are embodied as machine-executable instructions, these machine-executable instructions may be implemented in any suitable manner, including as a number of functional facilities, each providing one or more operations to complete execution of algorithms operating according to these techniques. A “functional facility,” however instantiated, is a structural component of a computer system that, when integrated with and executed by one or more computers, causes the one or more computers to perform a specific operational role. A functional facility may be a portion of or an entire software element. For example, a functional facility may be implemented as a function of a process, or as a discrete process, or as any other suitable unit of processing. If techniques described herein are implemented as multiple functional facilities, each functional facility may be implemented in its own way; all need not be implemented the same way. Additionally, these functional facilities may be executed in parallel and/or serially, as appropriate, and may pass information between one another using a shared memory on the computer(s) on which they are executing, using a message passing protocol, or in any other suitable way.

Generally, functional facilities include routines, programs, objects, components, data structures, etc., that perform particular tasks or implement particular abstract data types. Typically, the functionality of the functional facilities may be combined or distributed as desired in the systems in which they operate. In some implementations, one or more functional facilities carrying out techniques herein may together form a complete software package. These functional facilities may, in alternative embodiments, be adapted to interact with other, unrelated functional facilities and/or processes, to implement a software program application.

Some exemplary functional facilities have been described herein for carrying out one or more tasks. It should be appreciated, though, that the functional facilities and division of tasks described is merely illustrative of the type of functional facilities that may implement using the exemplary techniques described herein, and that embodiments are not limited to being implemented in any specific number, division, or type of functional facilities. In some implementations, all functionalities may be implemented in a single functional facility. It should also be appreciated that, in some implementations, some of the functional facilities described herein may be implemented together with or separately from others (e.g., as a single unit or separate units), or some of these functional facilities may not be implemented.

Machine-executable instructions implementing the techniques described herein (when implemented as one or more functional facilities or in any other manner) may, in some embodiments, be encoded on one or more computer-readable media, machine-readable media, etc., to provide functionality to the media. Computer-readable media include magnetic media such as a hard disk drive, optical media such as a CD or a DVD, a persistent or non-persistent solid-state memory (e.g., Flash memory, Magnetic RAM, etc.), or any other suitable storage media. Such a computer-readable medium may be implemented in any suitable manner. As used herein, the terms “computer-readable media” (also called “computer-readable storage media”) and “machine-readable media” (also called “machine-readable storage media”) refer to tangible storage media. Tangible storage media are non-transitory and have at least one physical, structural component. In a “computer-readable medium” and “machine-readable medium” as used herein, at least one physical, structural component has at least one physical property that may be altered in some way during a process of creating the medium with embedded information, a process of recording information thereon, or any other process of encoding the medium with information. For example, a magnetization state of a portion of a physical structure of a computer-readable medium, a machine-readable medium, etc., may be altered during a recording process.

Further, some techniques described above comprise acts of storing information (e.g., data and/or instructions) in certain ways for use by these techniques. In some implementations of these techniques—such as implementations where the techniques are implemented as machine-executable instructions—the information may be encoded on a computer-readable storage media. Where specific structures are described herein as advantageous formats in which to store this information, these structures may be used to impart a physical organization of the information when encoded on the storage medium. These advantageous structures may then provide functionality to the storage medium by affecting operations of one or more processors interacting with the information; for example, by increasing the efficiency of computer operations performed by the processor(s).

In some, but not all, implementations in which the techniques may be embodied as machine-executable instructions, these instructions may be executed on one or more suitable computing device(s) and/or electronic device(s) operating in any suitable computer and/or electronic system, or one or more computing devices (or one or more processors of one or more computing devices) and/or one or more electronic devices (or one or more processors of one or more electronic devices) may be programmed to execute the machine-executable instructions. A computing device, electronic device, or processor (e.g., processor circuitry) may be programmed to execute instructions when the instructions are stored in a manner accessible to the computing device, electronic device, or processor, such as in a data store (e.g., an on-chip cache or instruction register, a computer-readable storage medium and/or a machine-readable storage medium accessible via a bus, a computer-readable storage medium and/or a machine-readable storage medium accessible via one or more networks and accessible by the device/processor, etc.). Functional facilities comprising these machine-executable instructions may be integrated with and direct the operation of a single multi-purpose programmable digital computing device, a coordinated system of two or more multi-purpose computing device sharing processing power and jointly carrying out the techniques described herein, a single computing device or coordinated system of computing device (co-located or geographically distributed) dedicated to executing the techniques described herein, one or more FPGAs for carrying out the techniques described herein, or any other suitable system.

Embodiments have been described where the techniques are implemented in circuitry and/or machine-executable instructions. It should be appreciated that some embodiments may be in the form of a method, of which at least one example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Various aspects of the embodiments described above may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The phrase “and/or,” as used herein in the specification and in the claims, should be understood to mean “either or both,” of the elements so conjoined, e.g., elements that are conjunctively present in some cases and disjunctively present in other cases. Multiple elements listed with “and/or” should be construed in the same fashion, e.g., “one or more” of the elements so conjoined. Other elements may optionally be present other than the elements specifically identified by the “and/or” clause, whether related or unrelated to those elements specifically identified. Thus, as a non-limiting example, a reference to “A and/or B,” when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

The indefinite articles “a” and “an,” as used herein in the specification and in the claims, unless clearly indicated to the contrary, should be understood to mean “at least one.”

As used herein in the specification and in the claims, the phrase, “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently, “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” “having,” “containing,” “involving,” and variations thereof herein, is meant to encompass the items listed thereafter and equivalents thereof as well as additional items.

All definitions, as defined and used herein, should be understood to control over dictionary definitions, definitions in documents incorporated by reference, and/or ordinary meanings of the defined terms.

The word “exemplary” is used herein to mean serving as an example, instance, or illustration. Any embodiment, implementation, process, feature, etc., described herein as exemplary should therefore be understood to be an illustrative example and should not be understood to be a preferred or advantageous example unless otherwise indicated.

Having thus described several aspects of at least one embodiment, it is to be appreciated that various alterations, modifications, and improvements will readily occur to those skilled in the art. Such alterations, modifications, and improvements are intended to be part of this disclosure and are intended to be within the spirit and scope of the principles described herein. Accordingly, the foregoing description and drawings are by way of example only.

What is claimed is:

1. A method for triggering fundus imaging, comprising:
detecting a pupil of a subject in an image;
controlling at least one actuator to align an imaging path of a fundus camera with the pupil after detecting the pupil in the image; and
capturing a fundus image of a retina of the subject at a gaze angle after determining that the pupil is oriented towards an intended fixation target direction based on the gaze angle associated with the pupil in the image.

2. The method of claim 1, wherein the image is a stereo image, and the method further comprising:
capturing a first image from a first camera and a second image from a second camera;
processing the first image and the second image to form the stereo image; and
detecting the pupil in the first image and the second image to detect the pupil of the subject in the stereo image.

3. The method of claim 1, wherein the image comprises a first frame and a second frame, and the method further comprising performing at least one of edge detection or thresholding to determine whether the pupil is detected in at least one of the first frame or the second frame.

4. The method of claim 1, wherein the image comprises a first frame and a second frame, and the method further comprising:
executing a first machine-learning model using the first frame as at least one first input to generate at least one first output, the at least one first output representative of whether the pupil is detected in the first frame; and
executing a second machine-learning model, substantially in parallel with the executing of the first machine-learning model, using the second frame as at least one second input to generate at least one second output, the at least one second output representative of whether the pupil is detected in the second frame.

5. The method of claim 1, further comprising executing a machine-learning model using at least part of the image as at least one input to generate at least one output, the at least one output representative of whether the pupil is detected in the image.

6. The method of claim 5, wherein the executing of the machine-learning model comprises performing at least one of (i) concentric ellipse detection to detect at least one of an iris of the subject or the pupil in the image or (ii) performing line detection to detect an eyelid in the image, and the detection of the eyelid to be representative of whether an eye of the subject is closed.

7. The method of claim 1, further comprising performing one or more pre-processing operations on the image, and wherein the one or more pre-processing operations comprise at least one of removing glare from a portion of the image, correcting illumination on the portion of the image, or cropping the portion of the image.

8. The method of claim 1, wherein the pupil is a first pupil, and the method further comprising:
obtaining a plurality of images of second pupils, the plurality of images labeled with metadata;
training a machine-learning model using the plurality of images and the metadata;
compiling the machine-learning model into at least one of an executable file, machine-readable instructions, or a configuration image after determining that an accuracy of the machine-learning model satisfies a threshold; and
at least one of executing the executable file, executing the machine-readable instructions, or instantiating the configuration image to detect the first pupil of the subject in the image.

9. The method of claim 8, wherein the metadata is representative of at least one of an indication whether the second pupils are detected in respective ones of the plurality of images, a degree to which corresponding eyes of the second pupils are open or closed, or a gaze angle of respective ones of the second pupils.

10. The method of claim 1, wherein the image comprises a first frame, and the method further comprising:
executing a machine-learning model using the first frame as at least one input to generate at least one output, the at least one output representative of a determination of the gaze angle, the gaze angle being an angle with respect to the pupil and the fundus camera.

11. The method of claim 1, further comprising:
measuring a first value of a dimension of the pupil; and
generating a command to cause the subject to increase the dimension of the pupil after determining that the first value does not satisfy a threshold, the command comprising at least one of audible, tactile, or visual feedback to the subject.

12. The method of claim 1, further comprising:
measuring a first value of a dimension of the pupil;
determining at least one first coordinate of the pupil in a coordinate system associated with the fundus camera after determining that the first value satisfies a threshold; and
determining a correction to at least one second coordinate of the fundus camera after determining that the at least one first coordinate is not associated with a three-dimensional target zone for the pupil; and wherein
the controlling of the at least one actuator is based on the correction to the at least one second coordinate.

13. The method of claim 1, wherein the image comprises a first frame and a second frame, the gaze angle is a first gaze angle, and the method further comprising:
determining a ratio of a first width of the pupil in the first frame and a second width of the pupil in the second frame;
determining the first gaze angle based on the ratio; and
executing a machine-learning model using at least one of the first frame or the second frame as at least one input to generate at least one output representing a second gaze angle associated with the pupil, and wherein,
the determining that the pupil is oriented towards the target direction comprises determining that a difference between the first gaze angle and the second gaze angle satisfies a threshold.

14. The method of claim 1, further comprising:
determining that the pupil is not oriented towards the target direction;
changing the intended fixation target direction towards a direction in which the pupil is gazing; and
capturing the fundus image of the retina after the changing in the intended fixation target direction.

15. The method of claim 1, wherein capturing the fundus image of the retina is in response to detecting that an eyelid of the subject reopened.

16. The method of claim 1, wherein the at least one actuator comprises a first motor, a second motor, and a third motor, and the method further comprising at least one of:
controlling the first motor to move the fundus camera in a first direction;
controlling the second motor to move the fundus camera in a second direction orthogonal to the first direction; or
controlling the third motor to move the fundus camera orthogonal to at least one of the first direction or the second direction.

17. The method of claim 1, the method further comprising:
splitting the image into a first image portion and a second image portion using a prism configuration;
processing the first image portion and the second image portion to form a three-dimensional image; and
detecting the pupil in the three-dimensional image.

18. The method of claim 1, wherein the image comprises a first image and a second image, and the method further comprising:
determining the pupil is not in at least one of the first image or the second image; and
outputting feedback to the subject to cause the subject to move such that the pupil is detected in at least one of the first image or the second image.

19. The method of claim 18, wherein the feedback is at least one of audio feedback, haptic feedback, or visual feedback to the subject,
wherein the audio feedback comprises outputting audio using at least one speaker, the audio comprising audible instructions for the subject to move their head towards an instructed direction,
wherein the haptic feedback comprises outputting a vibration using at least one haptic actuator, the vibration to indicate to the subject to move their head towards an instructed direction, and
wherein the visual feedback comprises projecting an image using at least one display device, the image comprising instructions in natural language text for the subject to move their head towards an instructed direction.

20. At least one non-transitory computer-readable storage medium comprising instructions that, when executed, cause at least one processor to perform a method for triggering fundus imaging, comprising:
detecting a pupil of a subject in an image;
controlling at least one actuator to align an imaging path of a fundus camera with the pupil after detecting the pupil in the image; and
capturing a fundus image of a retina of the subject at a gaze angle after determining that the pupil is oriented towards an intended fixation target direction based on the gaze angle associated with the pupil in the image.

21. A fundus camera system comprising a three-dimensional visualization system, at least one memory storing machine-readable instructions, and at least one processor configured to execute the machine-readable instructions to perform at least a method for triggering fundus imaging, comprising:
detecting a pupil of a subject in an image;
controlling at least one actuator to align an imaging path of a fundus camera with the pupil after detecting the pupil in the image; and
capturing a fundus image of a retina of the subject at a gaze angle after determining that the pupil is oriented towards an intended fixation target direction based on the gaze angle associated with the pupil in the image.
